(19) United States
(12) Patent Application Publication
(10) Pub. No.: US 2026/0358882 A1
(43) Pub. Date: Nov. 12, 2026

(54) SYSTEM AND METHOD FOR PROCESSING MULTI-MODAL DATA UTILIZING MACHINE-LEARNING TO GENERATE CONTEXT-AWARE ADAPTIVE RESPONSES

(71) Applicant: State Farm Mutual Automobile Insurance Company, Bloomington, IL (US)

(72) Inventors: Edward W. Breitweiser, Bloomington, IL (US); Darcy Sementi, Tuscola, IL (US); Gregory Wong, New Albany, OH (US); Brian A. Steigerwald, Bloomington, IL (US); Kayla M. Welcome, Mesa, AZ (US); Steven B. Jenner, Atlanta, GA (US); Jennifer L. Crawford, Normal, IL (US)

(73) Assignee: State Farm Mutual Automobile Insurance Company, Bloomington, IL (US)

(21) Appl. No.: 19/211,695

(22) Filed: May 19, 2025

Related U.S. Application Data
(60) Provisional application No. 63/802,880, filed on May 9, 2025.

(51) Int. Cl. G06F 3/01; G06T 11/00; G16H 50/70

(52) U.S. Cl. CPC G06F 3/011; G06T 11/00; G16H 50/70; G06T 2200/24; G06T 2210/32

(57) ABSTRACT

Systems and methods for processing data to generate one or more context-aware adaptive responses are disclosed. The method may include, such as by one or more processors, transceivers, and/or sensors: (1) receiving data associated with a user from sensor(s); (2) processing the received data to transform the received data into a structured format; (3) inputting the processed data into a trained machine-learning model for generating the one or more context-aware adaptive responses for the user; (4) generating a display of the one or more context-aware adaptive responses and a corresponding avatar; (5) outputting the display and the avatar to a user interface of a device associated with the user; (6) monitoring one or more activities performed by the user based upon the display of the one or more context-aware adaptive responses; and/or (7) updating the avatar based upon the monitored user activities.

### CROSS-REFERENCE TO RELATED APPLICATION

This patent application claims the benefit of priority to U.S. Provisional Application No. 63/802,880, filed on May 9, 2025, the entirety of which is incorporated herein by reference.

### TECHNICAL FIELD

This present disclosure relates generally to the field of data processing, machine-learning, and data-driven recommendation systems. In particular, the present disclosure relates to processing multi-source sensor data associated with users for generating real-time responses using machine-learning techniques.

### BACKGROUND

Conventional health-related applications may suffer from fragmented data collection, static recommendations, and/or limited integration across diverse user contexts. Traditional systems may rely upon self-reported data or limited wearables integration, leading to incomplete health profiles and reactive recommendations rather than proactive, predictive insights. Additionally, conventional methods may lack dynamic personalization, as the methods may offer generic advice that may not adapt to real-time activities or evolving user habits.

In the context of integrating media habits, existing technologies may be technically challenged regarding assisting users through media consumption due to the lack of real-time behavioral triggers and/or personalized rule-based interventions. Traditional systems may not be designed to intercept the natural flow of sedentary entertainment consumption and/or dynamically adjust media content based upon the health goals of a specific user. Furthermore, conventional methods for tracking media interaction may be superficial, failing to leverage opportunities like commercial breaks for proactive health engagement or prompting the user to engage in healthy behavior before continuing sedentary media consumption.

Conventional driver behavior analysis systems may focus upon post-incident reporting, lacking real-time predictive intervention. For example, current telematics applications may be limited in the ability to synthesize sensor data into actionable, context-specific recommendations, such as selecting optimal parking for the health and safety of the user. The conventional systems may operate independently of personal lifestyle data and may lack the ability to tailor parking suggestions for specific users. Conventional techniques may include other ineffectiveness, encumbrances, inefficiencies, and drawbacks, as well.

### SUMMARY

The present embodiments may relate, inter alia, to the field of data processing, machine-learning, and data-driven recommendation systems, such as those discussed above and elsewhere herein. Specifically, the present computer systems and computer-implemented methods may solve technical challenges by integrating multi-modal data streams, (e.g., real-time sensor inputs, user-generated health, media activity data, vehicle and/or home telematics data, etc.) from diverse sources into a unified platform that may apply advanced machine-learning algorithm(s) to generate personalized, context-aware recommendations. The system may address challenges related to data heterogeneity, behavioral prediction, real-time engagement, and/or adaptive feedback by enabling seamless data normalization, predictive analytics, and/or automated interventions that may guide users towards healthier behaviors, improve safety, and/or increase adherence to preventive actions through dynamic content modulation.

In one aspect, a computer-implemented method for processing data to generate one or more context-aware adaptive responses may be provided. The computer-implemented method may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another and operate as input and/or output devices. In one instance, the computer-implemented method may be performed by one or more local or remote processors of a computing system in communication with one or more local or remote data sources.

The computer-implemented method may include, via one or more local or remote processors, transceivers, sensors, servers, and/or other components: (1) receiving, by the one or more processors, data associated with at least one user from one or more sensors; (2) processing, by the one or more processors, the received data to transform the received data into a structured format; (3) inputting, by the one or more processors, the processed data into a trained machine-learning model for generating the one or more context-aware adaptive responses for the at least one user; (4) generating, by the one or more processors, a display (and/or other output, such as verbal or audio output via a voice bot in addition to visual or graphical output) of the one or more context-aware adaptive responses and a corresponding avatar; (5) outputting, by the one or more processors, the display (and/or any audible output) and the avatar to a user interface of at least one device associated with the at least one user; (6) in response to the outputting, monitoring, by the one or more processors, one or more activities performed by the at least one user based upon the display of the one or more context-aware adaptive responses; and/or (7) updating, by the one or more processors, the avatar based upon the monitored one or more user activities. The method may include additional, less, or alternate functionality, including that discussed elsewhere herein.

In another aspect, a computer system for processing data to generate one or more context-aware adaptive responses may be provided. The computer system may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the computer system may be performed by one or more local or remote processors of a computing system in communication with one or more local or remote data sources, and at least one non-transitory computer readable medium storing instructions which, when executed by the one or more processors, cause the one or more processors to perform certain operations.

The computer system may include, via one or more processors, non-transitory computer readable medium, transceivers, sensors, servers, and/or other components to perform operations that may include: (1) receiving data associated with at least one user from one or more sensors; (2) processing the received data to transform the received data into a structured format; (3) inputting the processed data into a trained machine-learning model for generating the one or more context-aware adaptive responses for the at least one user; (4) generating a display (such as visual, graphical, audible, textual, verbal, and/or other output) of the one or more context-aware adaptive responses and a corresponding avatar; (5) outputting the display and the avatar to a user interface of at least one device associated with the at least one user; (6) in response to the outputting, monitoring one or more activities performed by the at least one user based upon the display of the one or more context-aware adaptive responses; and/or (7) updating the avatar based upon the monitored one or more user activities. The system may include additional, less, or alternate functionality, including that discussed elsewhere herein.

In yet another aspect, a non-transitory computer readable medium for processing data to generate one or more context-aware adaptive responses may be provided. The non-transitory computer readable medium may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the non-transitory computer readable medium may be performed by one or more local or remote processors of a computing system in communication with one or more local or remote data sources, and at least one non-transitory computer readable medium storing instructions which, when executed by the one or more processors, cause the one or more processors to perform certain operations.

The non-transitory computer readable medium may include, via one or more processors, transceivers, sensors, servers, and/or other components: (1) receiving data associated with at least one user from one or more sensors; (2) processing the received data to transform the received data into a structured format; (3) inputting the processed data into a trained machine-learning model for generating the one or more context-aware adaptive responses for the at least one user; (4) generating a display of the one or more context-aware adaptive responses and a corresponding avatar; (5) outputting the display (along with any graphical or audible output generated) and the avatar to a user interface of at least one device associated with the at least one user; (6) in response to the outputting, monitoring one or more activities performed by the at least one user based upon the display of the one or more context-aware adaptive responses; and/or (7) updating the avatar based upon the monitored one or more user activities. The operations may include additional, less, or alternate functionality, including that discussed elsewhere herein.

In one aspect, a computer-implemented method for dynamic integration of sensor data utilizing a trained machine-learning model for modifying media content may be provided. The computer-implemented method may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another and operate as input and/or output devices. In one instance, the computer-implemented method may be performed by one or more local or remote processors of a computing system in communication with one or more local or remote data sources.

The computer-implemented method may include, via one or more local or remote processors, transceivers, sensors, servers, and/or other components: (1) receiving, by the one or more processors, data associated with at least one user from one or more sensors; (2) transforming, by the one or more processors, the received data into a structured format; (3) inputting, by the one or more processors, the transformed data into the trained machine-learning model to identify one or more patterns within the data indicating a deviation from one or more predefined activity thresholds; (4) determining, by the one or more processors, a correlation between the media content displayed in at least one device and the deviation from the one or more predefined activity thresholds; (5) based upon the determined correlation, selecting, by the one or more processors, alternative content data for replacing or supplementing the media content displayed in the at least one device; and/or (6) modifying, by the one or more processors, the media content displayed in the at least one device to include the alternative content data. The method may include additional, less, or alternate functionality, including that discussed elsewhere herein.

In another aspect, a computer system for dynamic integration of sensor data utilizing a trained machine-learning model for modifying media content may be provided. The computer system may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the computer system may be performed by one or more local or remote processors of a computing system in communication with one or more local or remote data sources, and at least one non-transitory computer readable medium storing instructions which, when executed by the one or more processors, cause the one or more processors to perform certain operations.

The computer system may include, via one or more processors, non-transitory computer readable medium, transceivers, sensors, servers, and/or other components to perform operations that may include: (1) receiving data associated with at least one user from one or more sensors; (2) transforming the received data into a structured format; (3) inputting the transformed data into the trained machine-learning model to identify one or more patterns within the data indicating a deviation from one or more predefined activity thresholds; (4) determining a correlation between the media content displayed in at least one device and the deviation from the one or more predefined activity thresholds; (5) based upon the determined correlation, selecting alternative content data for replacing or supplementing the media content displayed in the at least one device; and/or (6) modifying the media content displayed in the at least one device to include the alternative content data. The system may include additional, less, or alternate functionality, including that discussed elsewhere herein.

In yet another aspect, a non-transitory computer readable medium for dynamic integration of sensor data utilizing a trained machine-learning model for modifying media content may be provided. The non-transitory computer readable medium may be implemented via one or more local or remote processors, servers, transceivers, memory units, mobile devices, voice bots or chatbots, ChatGPT bots, InstructGPT bots, Codex bots, Google Bard bots, and/or other electronic or electrical components, which may be in wired or wireless communication with one another. In one instance, the non-transitory computer readable medium may be performed by one or more local or remote processors of a computing system in communication with one or more local or remote data sources, and at least one non-transitory computer readable medium storing instructions which, when executed by the one or more processors, cause the one or more processors to perform certain operations.

The non-transitory computer readable medium may include, via one or more processors, transceivers, sensors, servers, and/or other components: (1) receiving data associated with at least one user from one or more sensors; (2) transforming the received data into a structured format; (3) inputting the transformed data into the trained machine-learning model to identify one or more patterns within the data indicating a deviation from one or more predefined activity thresholds; (4) determining a correlation between the media content displayed in at least one device and the deviation from the one or more predefined activity thresholds; (5) based upon the determined correlation, selecting alternative content data for replacing or supplementing the media content displayed in the at least one device; and/or (6) modifying the media content displayed in the at least one device to include the alternative content data. The operations may include additional, less, or alternate functionality, including that discussed elsewhere herein.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 depicts a diagram showing an exemplary architecture for a system that leverages advanced data collection, machine-learning, and real-time analytics for generating (i) personalized health assessments, (ii) optimizing media for behavior modification, and/or (iii) parking and driving safety recommendations, according to certain aspects of the disclosure.

FIG. 2A is an exemplary flowchart of a computer-implemented or computer-based process for processing data to generate one or more context-aware adaptive responses, according to one or more embodiments.

FIG. 2B depicts a diagram illustrating a system for multi-source data collection, real-time data processing, and machine-learning-based personalized recommendations, according to certain aspects of the disclosure.

FIG. 3A is an exemplary flowchart of a computer-implemented or computer-based process for dynamic integration of sensor data utilizing a trained machine-learning model for modifying media content, according to one or more embodiments.

FIG. 3B depicts a diagram illustrating a system for analyzing user behavior and media consumption patterns for generating personalized recommendations, according to certain aspects of the disclosure.

FIG. 4 depicts a diagram illustrating a system for analyzing real-time driving and environmental data for generating personalized recommendations, according to certain aspects of the disclosure.

FIG. 5 shows an exemplary machine-learning training flow chart, according to one or more embodiments.

FIG. 6 illustrates an implementation of an exemplary computer system that executes one or more techniques presented herein.

Advantages will become more apparent to those skilled in the art from the following description of the preferred embodiments that have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments may relate, inter alia, to computer systems and computer-implemented methods that may solve technical challenges by integrating diverse health, lifestyle, and/or biometric data sources into a unified framework, using machine-learning models to detect health risks and generate proactive, personalized health action plans. The present embodiments may also relate to methods that may dynamically adapt media and social media experiences through real-time monitoring and leveraging machine-learning models to optimize the timing and delivery of health-positive behavioral prompts. The present embodiments may further relate to methods that may fuse real-time driving signals, environmental context, and/or user preferences, using machine-learning models to deliver personalized driving and parking recommendations that may enhance safety and health outcomes.

Traditional systems may be limited by the system’s reliance upon static datasets, siloed device integrations, and/or reactive analytics. The conventional methods may collect fragmented user data, either through isolated wearable devices or sporadic self-reporting, which may lead to a significant gap in understanding the users’ overall health trajectory. These systems may lack robust pipelines to process heterogeneous data, such as biometric signals, lifestyle activities, and/or medical records, into a unified format suitable for predictive modeling. Since conventional systems are largely event-driven and reactive, they may provide generic recommendations based upon surface-level activity metrics, missing more nuanced patterns in user health behavior. As a result, conventional systems may be technically challenged to deliver timely, individualized health insights that may reflect the users’ changing lifestyle conditions.

Conventional technologies that attempt to integrate health and wellness promotion into digital media environments may suffer from inflexible architecture and limited behavioral responsiveness. Media consumption systems may be built to maximize user engagement, offering little support for health-oriented interventions that may dynamically adjust content based upon the health goals of the users. Furthermore, existing systems may have limited mechanisms for real-time monitoring of user activity during media interactions, such as detecting when a commercial break occurs or encouraging micro-activities during idle periods. Traditional platforms may lack integration between user task management tools and media consumption timelines, making it difficult to coordinate productive reminders or prompt timely behavior nudges without disrupting the user experience. These technical limitations may result in disconnected and ineffective wellness engagement strategies within traditional media and social platforms.

Traditional driving behavior monitoring systems may be designed for retrospective analysis, offering after-trips reports or basic behavioral scores rather than delivering immediate, actionable advice. These systems may lack the ability to process and interpret real-time driving data streams to provide location-specific or situation-specific recommendations, such as suggesting safer parking options based upon lighting or proximity to destinations that encourage walking. Data fusion from multiple in-vehicle sensors, navigation systems, and/or environmental contexts (e.g., lighting conditions, weather, or pedestrian activity) may be absent, making conventional solutions rigid and non-responsive to immediate driving environments. The inability to deliver contextual, real-time guidance may significantly limit the effectiveness of these systems in enhancing user safety, convenience, and/or healthy movement habits while driving.

### Exemplary Computer System

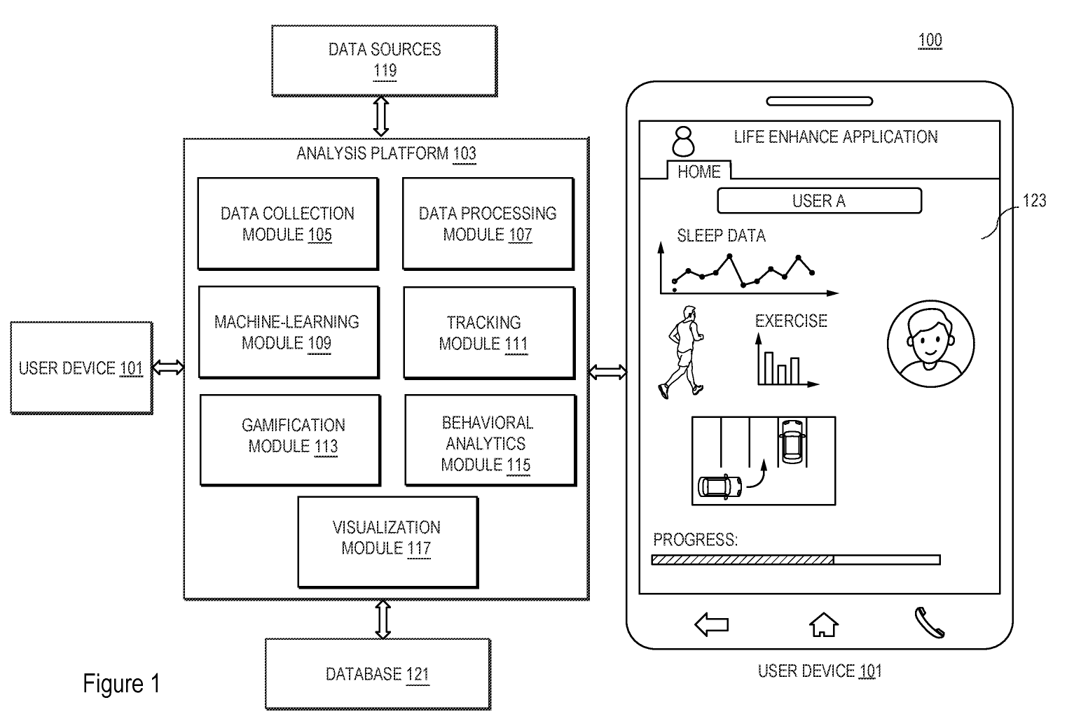

To address technical challenges, such as the above, system 100 of FIG. 1 may improve upon the conventional systems by implementing a unified data ingestion and normalization framework that may automatically integrate health, lifestyle, and/or biometric data from diverse sources into a standardized schema. The system 100 may continuously collect structured and/or unstructured data without relying upon excessive manual user input, thereby minimizing user burden and ensuring more complete health profiles. The system 100 may support secure interoperability with third-party wearable devices, sensory applications, and/or medical record systems through real-time APIs that may maintain regulatory compliance. The system 100 may utilize a machine-learning model that may process the aggregated data to identify subtle health risks, behavioral patterns, and/or lifestyle trends that may not be obvious through rule-based methods, allowing the system 100 to tailor action plans to user-specific profiles with higher precision.

In one instance, the system 100 may dynamically integrate media consumption timelines and social media platforms to adapt user content based upon individualized health and wellness goals. The system 100 may establish real-time monitoring mechanisms that detect contextual cues (e.g., commercial breaks or periods of inactivity) and trigger targeted wellness prompts (e.g., task completion suggestions) without disrupting the user’s core media experience. By leveraging event-driven architecture, the system 100 may recommend customizable behavioral rules that encourage users to complete health-positive actions before resuming certain media activities. The system 100 may also synchronize task management with media timelines, ensuring that overdue tasks and wellness objectives are intelligently prioritized during idle periods. In addition, the system 100 may utilize the machine-learning model to analyze user interaction patterns, media preferences, and/or behavior compliance history to optimize the timing, frequency, and/or type of recommendations delivered during media sessions.

In one instance, the system 100 may employ real-time data capture and analysis pipelines that process driving signals, vehicle telemetry or telematics data (including acceleration, braking, cornering, route, direction, heading, speed, and other data), and/or environmental context to provide actionable, location-specific guidance during trips. The system 100 may fuse data from multiple onboard sensors, navigation maps, and/or environmental indicators (e.g., lighting conditions, weather data, and/or pedestrian traffic) to dynamically recommend safer and/or health-promoting parking options based upon user-specific circumstances. Additionally, the system 100 may utilize the machine-learning model to evaluate ongoing and historical trip data to predict user-specific needs (e.g., preferred walking distance, risk tolerance based upon prior parking choices, or areas associated with higher safety), enhancing the relevance and precision of suggested driving and parking actions.

FIG. 1 depicts a diagram showing an exemplary architecture for a system that leverages advanced data collection, machine-learning, and real-time analytics for generating (i) personalized health assessments, (ii) optimizing media for behavior modification, and/or (iii) parking and driving safety recommendations, according to certain aspects of the disclosure. FIG. 1 may include the computing system 100 that may include a user device 101, an analysis platform 103, data sources 119, and/or a database 121. It should be understood that other implementations of computing system 100 may omit one or more of the foregoing components and/or may include additional components, as the case may be.

In one instance, the user device 101 may include a smartphone, a tablet, a wearable device, or any portable electronic device capable of communicating with external systems. The user device 101 may be operatively coupled to one or more sensors configured to monitor various physical, environmental, and/or behavioral parameters associated with the user. The one or more sensors may include, but are not limited to, motion sensors (e.g., gyroscope, accelerometers) for detecting user movement and physical activity, a sleep monitoring sensor for determining sleep patterns and duration, location sensors (e.g., GPS) for determining spatial movement pattern and location context, biometric sensors (e.g., heart rate monitors, body temperature sensors) for detecting pulse and heart rate variability, audio sensors (e.g., microphones) for detecting sounds associated with user behavior, a camera sensor for detecting facial expressions or eye tracking for attention monitoring, a screen usage detection sensor for tracking media consumptions patterns, and/or environmental sensors (e.g., air quality sensors, light sensors). In some instances, the user device 101 may further be connected to external wearable devices, such as smartwatches, smart glasses, or smart clothing, each comprising embedded sensors configured to monitor user activity, physiological conditions, and/or environmental factors (e.g., a pressure sensor embedded in smart clothing for monitoring posture and respiration). The collected sensor data may include activity levels, location information, biometric reading, environmental quality metrics, and/or audio data, which is transmitted to the analysis platform 103.

In one instance, the analysis platform 103 may include a platform with multiple interconnected components. The analysis platform 103 may include one or more servers, intelligent networking devices, computing devices, components, and/or corresponding software for processing multimodal data to generate personalized recommendations and context-aware interventions across health, media, and/or mobility domains.

In one instance, the analysis platform 103 may integrate heterogeneous data into a unified, adaptive intelligence framework that may continuously assess user well-being. The analysis platform 103 may address the technical challenge of fragmented data acquisition, where lifestyle metrics, environmental data, traffic data, and/or medical records may exist in disparate and often incompatible formats. The analysis platform 103 may perform real-time ingestion, normalization, and semantic alignment of time-series data, unstructured content, and self-reported inputs. The analysis platform 103 may utilize machine-learning models trained upon multimodal data to identify risk trajectories, negative health behaviors, and recommend proactive interventions. By creating longitudinal health profiles and dynamically adjusting recommendations based upon real-time data and evolving evidence, the analysis platform 103 may deliver precision health guidance that may evolve with the users.

In another instance, the analysis platform 103 may overcome the challenge of delivering timely, non-intrusive behavioral recommendations by embedding the recommendations seamlessly into users’ media consumption habits. Traditional health prompts may often disrupt the user experience or are simply ignored, but the analysis platform 103 may utilize a context-aware feedback system that may adapt to real-time activity levels, user goals, and/or digital interactions. By intelligently timing interventions, such as encouraging physical activity during commercial breaks or curating media content aligned with wellness goals, the analysis platform 103 may enhance user engagement without introducing friction. This dynamic, personalized approach to media-linked behavioral strategies based upon ongoing usage patterns may influence user behavior without creating friction.

In a further instance, the analysis platform 103 may integrate situational awareness with long-term behavior modeling, overcoming the challenge of transforming raw sensor data into anticipatory, personalized driving guidance. Moving beyond static, rule-based recommendations, the analysis platform 103 may analyze a user’s historical routes, parking preferences, and contextual factors (e.g., time of the data and neighborhood safety) to offer tailored suggestions, such as recommending walkable parking or preparing for new drivers. By intelligently balancing user safety, convenience, and wellness, the analysis platform 103 may deliver proactive advice while also respecting privacy and responsiveness constraints inherent in mobile environments.

In one instance, the analysis platform 103 may include a data collection module 105, a data processing module 107, a machine-learning module 109, a tracking module 111, a gamification module 113, a behavioral analytics module 115, and a visualization module 117, or any combination thereof. As used herein, terms such as “component” or “module” generally encompass hardware and/or software, e.g., that a processor or the like used to implement associated functionality. It is contemplated that the functions of these components are combined in one or more components or performed by other components of equivalent functionality.

In one instance, the data collection module 105 may include various software applications (e.g., data mining applications in Extended Meta Language (XML)) that automatically search for and return the relevant data. The data collection module 105 may be communicatively coupled to the user device 101, one or more wearable devices, and/or external data sources (e.g., data sources 119) to receive real-time and/or historical data associated with one or more users. The data collected by the data collection module 105 may include, but is not limited to, sensor data (e.g., movement patterns, sleep patterns, activity levels, location data, biometric readings, environmental conditions, driving data), user behavioral data (e.g., application usage patterns, online activity), and/or external contextual data (e.g., weather conditions, traffic conditions). The data collection module 105 may also receive user-provided inputs, such as self-reported (i) preferences, (ii) stress levels, (iii) health data, or (iv) driving conditions. The data collection module 105 may automate data extraction, ensuring continuous updates and validation to maintain data integrity. It should be understood that the data may be collected and processed by the system 100 if the users have consented to the use of their data for processing to generate recommendations.

In one instance, the data processing module 107 may organize, filter, and/or prepare the collected data for further analysis. The data processing module 107 may receive raw data from the data collection module 105, which may include sensor data, user behavioral data, contextual data, and/or self-reported data. The data processing module 107 may employ advanced algorithms to clean and preprocess the data, ensuring that the data is in a suitable format for analysis. In one example, data cleansing may include removing or correcting erroneous data (e.g., redundant, incomplete, or incorrect data) to create high-quality data. The data cleansing technique may also include a data enhancement technique, where data may be made more complete by adding related information. In another example, the data preprocessing techniques may include filtering out noise, handling missing values, and/or aggregating data to provide a comprehensive overview. Additionally, the data processing module 107 may normalize the data into a consistent format and structure suitable for downstream analysis.

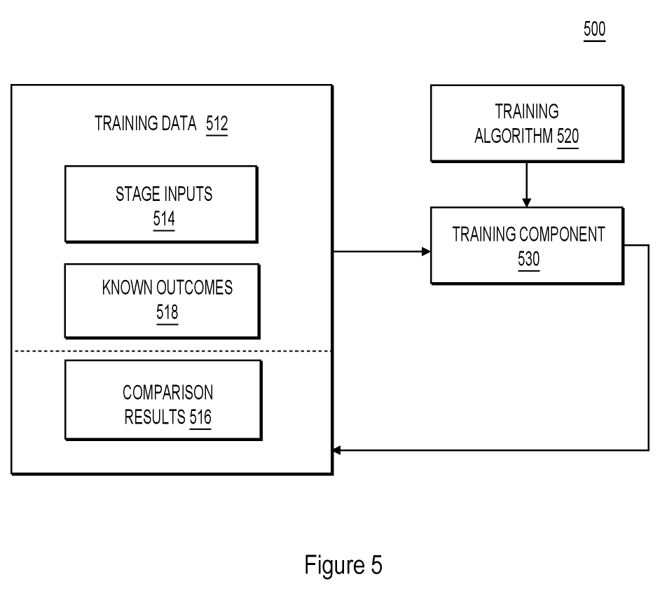

In one instance, the machine-learning module 109 may be configured for supervised machine learning, utilizing training data (e.g., training data 512 illustrated in the training flow chart of FIG. 5). The trained model may be configured for processing real-time and/or historical data, such as sensor data (e.g., movement patterns, sleep patterns, activity levels, location data, biometric readings, environmental conditions), user behavioral data (e.g., application usage patterns, online activity, driving data), and/or external contextual data (e.g., weather conditions, traffic conditions) to learn patterns associated with the user (e.g., physical health and mental well-being, driving behavior, media usage patterns, etc.). In one example, the machine-learning module 109 may perform model training using training data (e.g., data from other modules, that contain input and correct output, to allow the model to learn over time). The training may be performed based upon the deviation of a processed result from a documented result when the inputs are fed into the machine-learning model (e.g., an algorithm measures its accuracy through the loss function, adjusting until the error has been sufficiently minimized). The trained model may utilize exponential smoothing, autoregressive integrated moving average (ARIMA), or long short-term memory (LSTM) neural networks to analyze one or more features associated with users to identify behavioral patterns, driving patterns, detect trends in physical and mental well-being, assess deviations from expected behavior, predict potential risks, and/or generate personalized recommendations.

In one instance, the machine-learning module 109 may randomize the ordering of the training data, visualize the training data to identify relevant relationships between different variables, identify any data imbalances, and/or split the training data into two parts, where one part may be for training a model and the other part may be for validating the trained model, de-duplicating, normalizing, and/or correcting errors in the training data, and so on. The machine-learning module 109 may implement various machine learning techniques, e.g., neural network (e.g., recurrent neural networks, graph convolutional neural networks, deep learning neural networks), decision tree learning, random forest, association rule learning, inductive programming logic, K-nearest neighbors, cox proportional hazards model, support vector machines, Bayesian models, Gradient boosted machines (GBM), LightGBM (LGBM), Xtra tree classifier, etc. Implementation of the machine-learning module 109 is discussed in detail below.

In one instance, the machine-learning module 109 may process multidimensional health and lifestyle data collected from one or more users to build personalized health profiles and risk assessments. By analyzing inputs, such as physical activity, biometric readings, consumer behavior, sleep patterns, online activities, and/or medical history data, the machine-learning module 109 may identify negative health behaviors and predict users at elevated risk for chronic conditions. The machine-learning module 109 may classify one or more users into risk categories and may suggest personalized action plans, such as annual physicals, cancer screening reminders, or nicotine replacement strategies. The machine-learning module 109 may continuously retrain itself based upon user interaction and health outcomes, refining its predictive capabilities to ensure that health insights remain timely, actionable, and aligned with each user’s evolving needs.

In another instance, the machine-learning module 109 may analyze media consumption habits, social media activity patterns, and/or health goals to create dynamic models of user behavior and engagement. By leveraging natural language processing (NLP) techniques and activity recognition models, the machine-learning module 109 may classify content that may either positively or negatively impact user health behaviors. Reinforcement learning strategies may be employed to optimize the timing and type of content interventions, adjusting social media feeds to prioritize motivational posts about fitness, healthy eating, and/or mental wellness. During media viewing sessions, the machine-learning module 109 may identify optimal moments for nudging users, such as commercial breaks, and recommend actionable tasks like stretching or walking. The machine-learning module 109 may adapt recommendations based upon observed user compliance and preferences, ensuring an increasingly personalized and effective behavior modification experience.

In a further instance, the machine-learning module 109 may ingest real-time driving data and historical trip patterns to develop predictive models that may enhance driving safety and promote healthier behaviors. Through pattern recognition and anomaly detection techniques, the machine-learning module 109 may identify unsafe driving habits (e.g., harsh braking, excessing speed) and recommend corrective actions. Before each trip, the machine-learning module 109 may anticipate user needs based upon past behavior and environmental conditions, and may suggest safer, more efficient routes and well-lit parking areas to encourage both physical safety and incidental physical activity (e.g., walking from a distant parking spot). In addition, the machine-learning module 109 may predict life events, such as when a family member may be nearing driving age, offering proactive educational resources and incentives to prepare new drivers. By learning from every driving session, the machine-learning module 109 may ensure continuous improvement in both safety and user-specific guidance.

In one instance, the tracking module 111 may monitor and record user actions in response to the generated recommendations. The tracking module 111 may continuously collect data related to the user activities and/or behavior following the issuance of recommendations by the machine-learning module 109. This may include detecting whether the user followed the prompt to schedule a health screening, complete a suggested physical activity, improve dietary habits, and/or improve driving habits. The tracking module 111 may log real-time behavior data, interaction timestamps, location context, and outcome measures, which may be useful for evaluating the effectiveness of personalized recommendations. The tracking module 111 may correlate the recorded user actions with the original recommendation to assess adherence and responsiveness. Additionally, the tracking module 111 may analyze temporal patterns to determine short-term and long-term behavioral changes, enabling the system 100 to measure the effectiveness of recommendations over time. The tracking module 111 may compare the collected data to predefined adherence criteria that may correspond to expected behaviors or actions associated with the recommendations.

In one instance, the tracking module 111 may track the progression over a predetermined time period to determine one or more trends indicative of positive or negative changes in physical or mental well-being of the users. Based upon this analysis, the machine-learning module 109 may recalibrate the trained machine-learning models by adjusting one or more model parameters, retraining the trained machine-learning models with updated data, and/or incorporating new behavioral patterns observed in the user’s data. This dynamic recalibration may allow the machine-learning models to adapt to the user’s evolving needs and circumstances, ensuring that future recommendations remain relevant, effective, and personalized. For example, if a user consistently ignores certain types of recommendations, the recalibrated models may reduce the frequency of such prompts and instead focus upon alternative strategies more likely to encourage positive behavior.

In one instance, the gamification module 113 may facilitate user engagement and adherence by integrating game-like elements. The gamification module 113 may translate health goals, behavior changes, and/or safety practices into interactive challenges, reward systems, and/or progress-tracking mechanisms. In one example, users may earn points, badges, or status levels for completing activities, such as participating in preventive health screenings, achieving daily step goals, responding to wellness prompts during media breaks, or adopting safer driving habits. These achievements may be visually tracked through leaderboards or personal dashboards, encouraging continued participation through positive reinforcement and social comparison. The gamification module 113 may also link with partner ecosystems, offering real-world rewards, such as discounts upon healthy meals or fitness gear, to reinforce healthy behaviors. By creating a sense of progression, competition, and tangible reward, the gamification module 113 may turn routine tasks into compelling incentives, driving sustained user engagement and contributing to long-term lifestyle improvements.

In another instance, the gamification module 113 may generate visual and interactive representations of the user (e.g., avatar), that may evolve based upon the user’s activities, health behaviors, and/or engagement within the application. This avatar may reflect the user’s progress by visually adapting to healthy lifestyle choices, for example, becoming more energetic, vibrant, or gaining accessories and animations as the user completes goals like consistent physical activity, improved sleep, or following through with preventive care recommendations. Conversely, a lack of engagement or negative health trends may cause the avatar to appear fatigued or lose enhancements, serving as a gentle, non-intrusive form of feedback. The evolving avatar may foster a sensor of identity, ownership, and/or accountability, encouraging users to maintain or improve their health behaviors to see positive changes in their digital representations.

In one instance, the behavioral analytics module 115 may analyze user interaction patterns, behavioral trends, and/or engagement metrics generated across the system, including from activity tracking, recommendation acceptance, and gamification outcomes. The behavioral analytics module 115 may identify which interventions are most effective for different user segments, detect behavioral stagnation or drop-off points, and help personalize future recommendations based upon past behavioral responsiveness. By segmenting users and scoring the effectiveness of different notifications, the behavioral analytics module 115 may enable continuous behavioral optimization. The behavioral analytics module 115 may serve as a critical intelligence layer that may inform how content, gamified challenges, avatars, and/or visual outputs should be adapted for maximum impact.

In one example, the behavioral analytics module 115 may reveal that users may be more likely to complete health tasks when prompted during lunch breaks rather than in the evening. The system 100 may adjust notifications and challenges to coincide with lunch breaks, ensuring higher completion rates for activities like walking or hydration reminders. In another example, the behavioral analytics module 115 may determine that users respond better to interactive video-based wellness content rather than text-based articles. The system 100 may prioritize video tutorials for fitness exercises or stress-reduction techniques, ensuring content delivery in a format the user finds more engaging. In a further example, the behavioral analytics module 115 may determine that users who see friends or family members engaged in wellness challenges are more likely to participate themselves. The system 100 may send reminders to join a shared challenge, incorporating social features like leaderboards or group milestones to encourage collective participation. In another example, the behavioral analytics module 115 may determine that users are more likely to participate in health challenges if the users are located near health-related services, such as a gym or park. The system 100 may offer location-based rewards or reminders, encouraging users to visit a nearby wellness center or walk to a local park as part of their daily activity challenges.

In one instance, the visualization module 117 may convert the recommendations generated by the machine-learning module 109 into user-friendly visual, aural, and/or textual prompts. The visualization module 117 may employ various application programming interfaces (APIs) or other function calls corresponding to the application upon the user device 101, thus enabling the display of graphics primitives such as graphs, edges, icons, menus, buttons, data entry fields, etc. In one example, the visualization module 117 may enable a presentation of a graphical user interface (GUI) in the user device 101 that may facilitate visualization of one or more recommendations (e.g., display 123). In one example, display 123 may generate sleep data by displaying detailed graphs that track users’ sleep patterns over time, highlighting trends and offering insights into sleep quality and duration. In another example, display 123 may visualize exercise data, showing progress through interactive graphs that may track physical activity, such as steps taken or calories burned. In a further example, display 123 may include an avatar, reflecting changes in the user’s health and activity levels, such as improved fitness or increased engagement with wellness behaviors. In another example, display 123 may present parking recommendations, offering visual suggestions for optimal parking spots based upon factors like proximity to the destination or safety features, such as lighting and ease of access.

The visualization module 117 may ensure that the information is clear, timely, and personalized, enhancing the user’s ability to make informed decisions. The visualization module 117 may also comprise a variety of interfaces, for example, interfaces for data input and output devices, referred to as I/O devices, storage devices, and the like. Still further, the visualization module 117 may be configured to operate in connection with augmented reality (AR) processing techniques, wherein various applications, graphic elements, and features interact.

In one instance, the system 100 may be configured to retrieve supplemental data from data sources 119 for additional context. In one instance, the data sources 119 may include environmental databases that may provide real-time weather data, air quality data, and/or pollen levels that may facilitate tailoring suggestions for outdoor activities (e.g., good weather for a run) or indoor activities (e.g., poor air quality for users with respiratory conditions). In another instance, the data sources 119 may include traffic databases that may provide real-time traffic data for generating recommendations, such as suggesting walking or cycling routes based upon traffic congestion, promoting safe driving behavior by alerting users of high-risk traffic zones, and/or recommending alternative commute patterns for physical activity. In a further instance, the data sources 119 may include electronic health records (EHRs), user’s medical reports, and/or personal health history data, subject to user consent and privacy protections. The health data may include information about pre-existing medical conditions, prior diagnoses, medication history, allergies, or mental health assessments, which may facilitate tailoring recommendations specific to the user’s physical and mental health needs. In another instance, data sources 119 may include crime index databases that may facilitate the system 100 in modifying physical activity routes based upon lighting, safety, and neighborhood security, altering users to unsafe walking or driving conditions. It should be understood that the data sources 119 may include any other databases that provide relevant information pertaining to the users.

In one instance, the database 121 may include any type of database, such as relational, hierarchical, object-oriented, and/or the like, wherein data are organized in any suitable manner, including data tables or lookup tables. In one instance, the database 121 may store content associated with the users (e.g., policyholders), the user device 101, and the analysis platform 103, and may manage multiple types of information that aid in the content provisioning and sharing process. For example, the database 121 may store raw user data collected from the user device 101, sensors, and/or external data sources. Additionally, the database 121 may store processed data, such as data indicative of the user’s physical health, mental well-being, driving, and behavioral patterns, as well as user profiles, historical trends, and adherence records.

In another instance, the database 121 may include a machine-learning based training database with a pre-defined mapping defining a relationship between various input parameters and output parameters based upon various statistical methods. For example, the training database may include machine-learning algorithms to learn mappings between input parameters related to the users. In one example, the training database may include a dataset that includes data collections that are not subject-specific, e.g., data collections based upon population-wide observations, local, regional, or super-regional observations, and the like. The training database may be routinely updated and/or supplemented based upon the machine-learning methods.

The various elements of the computing system 100 may communicate with each other through a communication network. In one instance, the user device 101 may include a network detection sensor for detecting wireless signals or receivers for different communications (e.g., Bluetooth, Wi-Fi, Li-Fi, near field communication (NFC), etc.) from the communication network. The communication network may support a variety of different communication protocols and communication techniques.

In one instance, the communication network may allow the user device 101 (or one or more user devices) to communicate with the analysis platform 103. The communication network may include one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

The above presented modules and components of the analysis platform 103 may be implemented in hardware, firmware, software, or a combination thereof. Though depicted as a separate entity in FIG. 1, it is contemplated that the analysis platform 103 may be implemented for direct operation by the respective user device 101. As such, the analysis platform 103 may generate direct signal inputs by way of the operating system of the user device 101. In one instance, one or more of the modules 105-117 may be implemented for operation by the respective user device 101, as the analysis platform 103. The various executions presented herein contemplate any and all arrangements and models.

### Exemplary Machine-Learning Based Health Optimization Flowchart

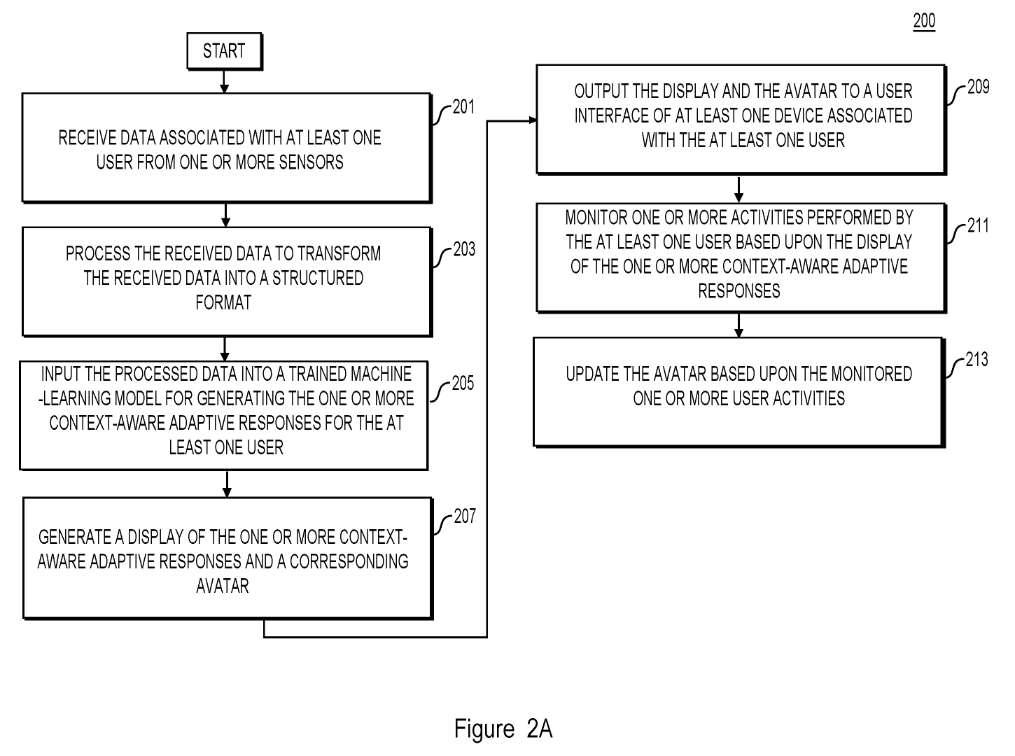

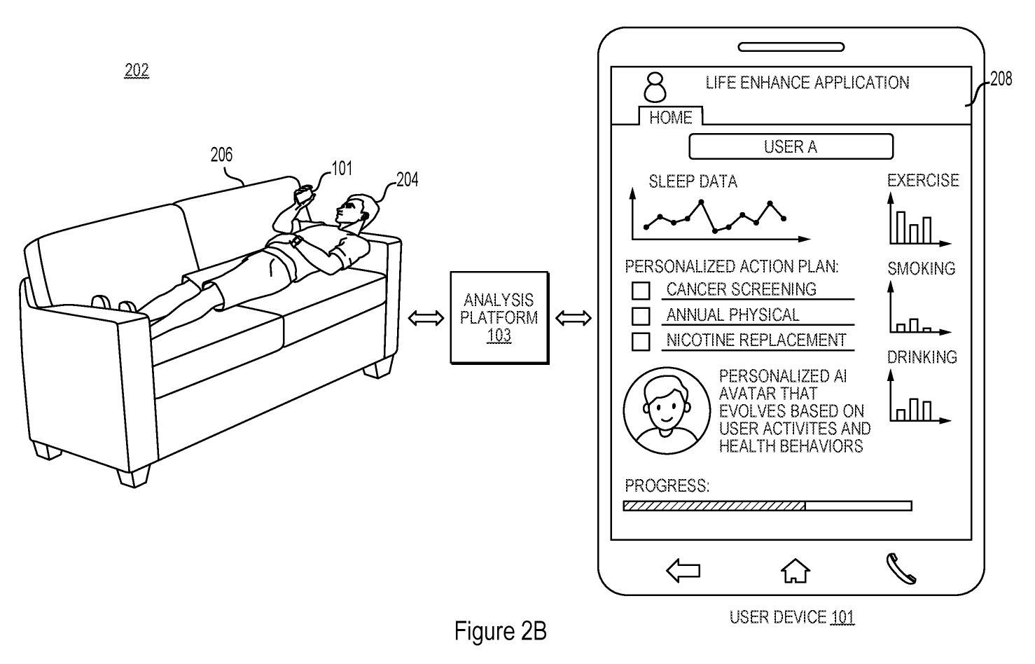

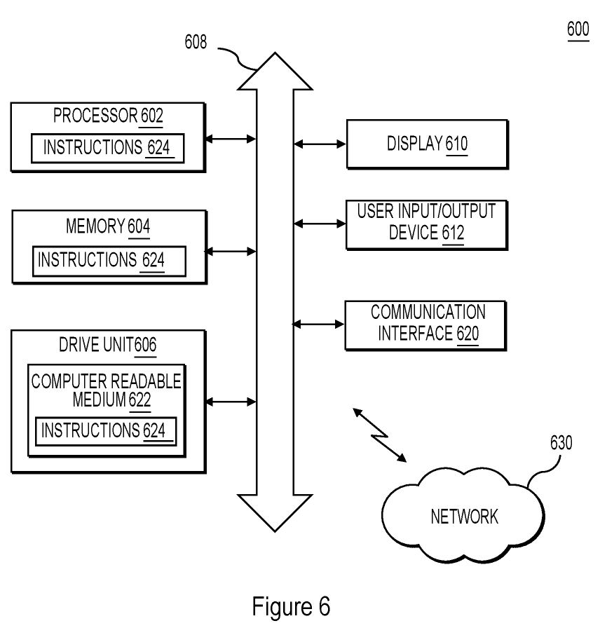

FIG. 2A is an exemplary flowchart of a computer-implemented or computer-based process for processing data to generate one or more context-aware adaptive responses, according to one or more embodiments. In one instance, the analysis platform 103 and/or any of the modules 105-117 may perform one or more portions of the process 200 and are implemented using, for instance, a chip set including a processor (e.g., processor 602) and a memory (e.g., memory 604), as shown in FIG. 6. As such, the analysis platform 103 and/or any of the modules 105-117 may be configured to facilitate accomplishing various parts of the process 200, as well as accomplishing embodiments of other processes described herein in conjunction with other components of the computing system 100. Although the process 200 is illustrated and described as a sequence of actions, operations, and/or functionality, it is contemplated that various embodiments of the process 200 may be performed in any order or combination and need not include all of the illustrated actions, operations, and/or functionality.

In block 201, the analysis platform 103 may receive data associated with a user from various sensors. The data associated with the user may include (i) behavioral data (e.g., exercise frequency, sleep habits, smoking habits, alcohol consumption, etc.), (ii) physiological data (e.g., heart rate, body temperature, respiratory rate, blood glucose level, etc.), (iii) contextual data (e.g., weather conditions, time of day, location data, etc.), (iv) historical data (e.g., past medical checkups, previous lifestyle habits, long-term trends in activity or sleep, previous interventions and outcomes, etc.), and/or (v) health data (e.g., electronic medical records, lab test results, health insurance claims, vaccination history, etc.). The sensors may include (i) a motion sensor (e.g., accelerometer, gyroscope, etc.) for detecting user movement and/or physical activity, (ii) a heart rate sensor (e.g., electrocardiography (ECG), photoplethysmography (PPG), etc.) for detecting pulse and heart rate variability, (iii) a sleep monitoring sensor for determining sleep patterns and duration, (iv) a temperature sensor for detecting changes in body temperature, (v) a microphone for detecting sounds associated with user behavior, (vi) a geolocation sensor (e.g., GPS) for determining spatial movement pattern and location context, and/or (vii) a pressure sensor embedded in smart clothing for monitoring posture and respiration.

In block 203, the analysis platform 103 may process the received data to transform the received data into a structured format. In one instance, the analysis platform 103 may (i) normalize one or more measurement units across multiple data sources to a common scale (e.g., convert recorded weights from pounds into kilograms to ensure consistency across user health profiles), (ii) align time-series data for consistency across sampling intervals (e.g., synchronize heart rate data from different sensors to 5-minute intervals to enable accurate trend analysis), and/or (iii) map unstructured data into a predefined data schema (e.g., extract relevant health metrics from uploaded medical PDFs and populate the corresponding fields in the user’s structured electronic health record).

In block 205, the analysis platform 103 may input the processed data into a trained machine-learning model for generating context-aware adaptive responses for the user. The context-aware adaptive responses may include (i) an activity suggestion (e.g., taking a 15-minute walk after lunch, performing guided stretching exercise in the morning, etc.), (ii) one or more personalized health plans (e.g., customized nutrition and exercise plans based upon BMI trends, etc.), (iii) one or more stress management techniques (e.g., guided meditation sessions, breathing exercise, etc.), (iv) one or more sleep optimization strategies (e.g., recommending consistent bedtimes based upon detected sleep irregularities and promoting blue light reduction before sleep, etc.), (v) one or more substance use reduction plans (e.g., nicotine replacement therapy and motivational reminders triggered by smoking patterns, etc.), and/or (vi) one or more preventive healthcare screenings (e.g., reminders to schedule annual physicals, cancer screening, or cholesterol tests based upon age and risk factors, etc.).

In one instance, the trained machine-learning model may analyze the processed data to extract statistical features (e.g., average daily step count, variance in heart rate, BMI trends, frequency of missed medications, etc.), temporal features (e.g., changes in sleep durations over weeks, time-of-day eating habits, seasonal activity fluctuations, progression of biometric indicators over time, etc.), and/or contextual features (e.g., correlations between stress levels and calendar events, sedentary behavior during workday versus weekends, etc.).

The trained machine-learning model may identify pattern(s) corresponding to the extracted features (e.g., a consistently elevated heart rate combined with low physical activity, a gradual decline in sleep quality over the past 30 days, increased junk food consumption during weekends, etc.). These patterns may be indicative of behavioral profiles, temporal health state transitions, or deviations from normative lifestyle baselines. The trained machine-learning model may generate prediction(s) based upon the pattern(s). The prediction(s) may include (i) dynamically inferred user states (e.g., the user is in a high-stress state based upon reduced sleep and/or elevated health rate, etc.), (ii) estimated progression of behavioral trajectories (e.g., continued decline in physical activity over the next two weeks if current trend persists, etc.), or (iii) probability score for possible interventions (e.g., a 70% probability that enrolling the user in a digital sleep coaching program may improve sleep quality within 30 days, etc.). The trained machine-learning model may select one or more of the context-aware adaptive response(s) based upon the prediction(s). In one instance, the trained machine-learning model may (i) recommend a relaxation technique when it predicts a high-stress state, (ii) suggest a physical activity break upon detecting prolonged sedentary behavior, (iii) suggest a healthy meal recipe upon predicting a pattern of unhealthy eating, prompt a user to go for a walk upon detecting low activity levels over the weekend.

In block 207, the analysis platform 103 may generate a display of the context-aware adaptive responses and a corresponding avatar. In one instance, the avatar may include a computer-generated, animated character configured to output visual and auditory cues based upon context data received from one or more sensors. The analysis platform 103 may generate notification(s) in the user interface of the user device 101 to encourage adherence to the context-aware adaptive responses. The notification(s) may include (i) one or more rewards, (ii) one or more premium discounts, (iii) one or more achievement-based badges, (iv) one or more goal-setting challenges, and/or (v) enhancing the avatar to reflect progress.

In block 209, the analysis platform 103 may output the display of the context-aware adaptive responses and the avatar in a user interface of a device associated with the user (e.g., user device 101). In one instance, the avatar may include customizable in appearance and personality to increase user relatability and engagement. The avatar may also display subtle visual cues or emotional expressions to reinforce positive behaviors. It may use voice, animation, or gesture-based communication to deliver recommendations in an engaging manner.

In block 211, the analysis platform 103 may monitor activities performed by the user based upon the display of the context-aware adaptive responses. The analysis platform 103 may receive real-time data streams from the sensors (e.g., sensors associated with user device 101). The analysis platform 103 may analyze the real-time data streams to determine a level of compliance to the context-aware adaptive responses using a rule-based analysis. The rule-based analysis may include predefined thresholds for the activities to assess the level of compliance (e.g., the analysis platform 103 may identify whether the user completed a recommended walk within 30 minutes of sedentary alert).

In block 213, the analysis platform 103 may update the avatar based upon monitored user activities. In one instance, the analysis platform 103 may dynamically adjust the appearance and characteristics of the avatar based upon improvement or deterioration in the condition of the user. The appearance and the characteristics may reflect a success or a failure in completing the context-aware adaptive responses. The analysis platform 103 may modify the facial expressions, posture, and/or clothing item of the avatar to visually indicate success or failure. In one instance, the success may be represented by positive traits including upright posture, happy facial expressions, and/or athletic clothing item in bright colors. In one instance, the failure may be represented by negative traits including a slouched posture, sad facial expressions, and/or untidy clothing item in dark colors.

### Exemplary Health Monitoring and Recommendation Generation System

FIG. 2B depicts a diagram illustrating a system for multi-source data collection, real-time data processing, and machine-learning-based personalized recommendations, according to certain aspects of the disclosure. In a comprehensive health monitoring environment 202, a user 204 is relaxing upon a sensor-embedded sofa 206 while interacting with a mobile device. The user is wearing smart clothing and a smartwatch, each equipped with an array of biometric sensors. The sensor-embedded sofa 206 may collect data upon pressure distribution, seating posture, and/or duration of sedentary behavior which may be processed to infer physical fatigue or stress level. The mobile device may collect contextual and behavioral data, such as application usage frequency, screen time, geolocation, ambient light and noise levels, and user-input lifestyle (e.g., dietary intake or self-reported symptoms). The smart clothing and smartwatch may collect continuous physiological signals, such as heart rate variability, respiratory rate, skin temperature, movement data, posture data, etc. All of these heterogeneous data are continuously aggregated and securely transmitted to the analysis platform 103.

The analysis platform 103 may incorporate behavioral and lifestyle variables derived from the user’s interaction with the smartphone application and connected devices. The behavioral and lifestyle variables may include exercise frequency, dietary patterns, tobacco and alcohol consumption, and/or transaction data indicating health-related purchasing behavior (e.g., gym memberships, fast food, or pharmacy visits). The analysis platform 103 may utilize a trained machine-learning model for parsing structured inputs (e.g., check-in logs or purchase history) and/or unstructured data (e.g., journal entries or social activity) to extract meaningful lifestyle indicators. The trained machine-learning model may classify risk levels, identify deviations from lifestyle baselines, dynamically update the user’s health profile, and/or calibrate recommendations.

The analysis platform 103 may receive and process the incoming data streams from the user’s connected devices, applying temporal alignment and contextual interpretation to generate meaningful insights. Sleep-related data collected from the smartwatch and smart clothing may be analyzed to identify trends and deviations from baseline health patterns. In this scenario, the analysis platform 103 may detect that the user consistently sleeps fewer hours than the normative benchmark based upon population-level data. This deviation may be flagged as a potential health risk and may contribute to the user’s evolving health profile, forming the basis for future recommendations and interventions.

The analysis platform 103 may generate a personalized action plan tailored to the user’s current risk profile and lifestyle context. This may include reminders and scheduling tools for preventive screenings, such as mammograms, colonoscopies, or annual physical exams. In addition, the analysis platform 103 may recommend targeted therapies, such as a nicotine replacement program if smoking behavior is detected. The analysis platform 103 may optimize these suggestions by balancing urgency, user readiness, and/or prior behavioral response to similar interventions. These recommendations may be presented within an intuitive and visually engaging user interface 208 upon the user device 101.

To enhance user engagement and facilitate long-term behavioral change, the analysis platform 103 may deploy an adaptive AI avatar in the user interface 208. This avatar may be dynamically generated using reinforcement learning techniques and may evolve based upon real-time user data. The appearance, tone, and/or content delivery of the avatar may be dynamically adapted according to the user’s compliance with action plans, behavioral milestones achieved, and/or overall health trajectory. For instance, if the user successfully increases the user’s average sleep duration and reduces tobacco intake, the avatar’s visual representation and demeanor may shift to reflect improved vitality and confidence. Health milestones, such as completing a preventive screening, may be celebrated with avatar-based animations and feedback loops that positively reinforce continued engagement.

In one instance, by fusing biometric, behavioral, and/or contextual data, the comprehensive health monitoring environment 202 may construct a rich, continuous health profile unique to each user. The modular design of the comprehensive health monitoring environment 202 may support scalability, enabling seamless integration of new data types or algorithms. The machine-learning model may ensure continuous adaptation by refining recommendations based upon evolving user behavior. The dynamic AI avatar may act as an intuitive feedback layer, transforming raw analytics into actionable, user-friendly insights. Together, these elements may create a responsive and intelligent platform that transitions from passive health tracking to proactive, personalized care.

### Exemplary User Activity Deviation Detections and Response Flowchart

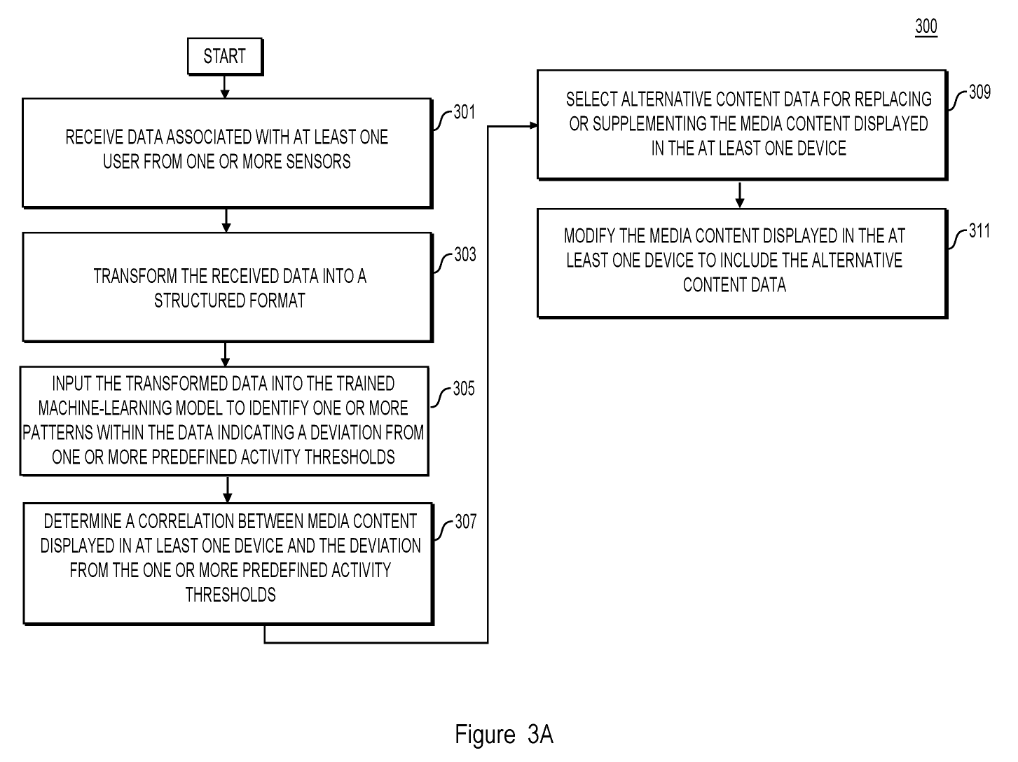

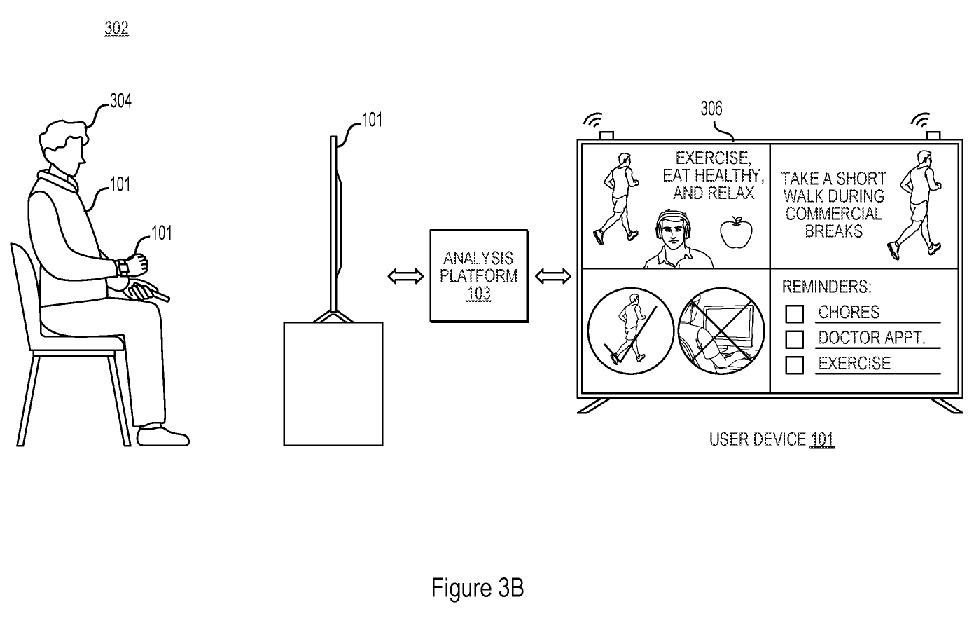

FIG. 3A is an exemplary flowchart of a computer-implemented or computer-based process for dynamic integration of sensor data utilizing a trained machine-learning model for modifying media content, according to one or more embodiments. In one instance, the analysis platform 103 and/or any of the modules 105-117 may perform one or more portions of the process 300 and are implemented using, for instance, a chip set including a processor (e.g., processor 602) and a memory (e.g., memory 604), as shown in FIG. 6. As such, the analysis platform 103 and/or any of the modules 105-117 may be configured to facilitate accomplishing various parts of the process 300, as well as accomplishing embodiments of other processes described herein in conjunction with other components of the computing system 100. Although the process 300 is illustrated and described as a sequence of actions, operations, and/or functionality, it is contemplated that various embodiments of the process 300 may be performed in any order or combination and need not include all of the illustrated actions, operations, and/or functionality.

In block 301, the analysis platform 103 may receive data associated with a user from sensor(s) (e.g., sensors associated with the user device 101). The data associated with the user may include (i) activity tracking data (e.g., step counts, sedentary time, exercise duration, etc.), (ii) media consumption data (e.g., hours of television watched, types of shows streamed, time spend upon social media application, etc.), (iii) task completion records (e.g., logged completion of wellness tasks, such as hydration reminders, medication intake, or stretching exercise, etc.), (iv) predetermined health related goals (e.g., walk 10,0000 steps daily, mediate 10 minutes per day, avoid sugar for one week, etc.), and/or (v) historical adherence data to the predefined activity threshold(s) (e.g., number of days user met daily step goals over past month, frequency of exercise session per week, etc.). The sensor(s) may include (i) a motion sensor (e.g., accelerometer, GPS sensor, etc.) for detecting user movement and physical activity, (ii) a heart rate sensor (e.g., photoplethysmography (PPG), etc.) for monitoring cardiovascular activity, (iii) a pressure sensor (e.g., barometric sensors, etc.) embedded in smart clothing for monitoring posture and respiration, (iv) a camera sensor for detecting facial expressions or eye tracking for attention monitoring, and/or (v) a screen usage detection sensor (e.g., software-based screen activity tracker in smart television) for tracking media consumptions patterns.

In block 303, the analysis platform 103 may transform the received data into a structured format. In one instance, transforming the received data into the structured format may include (i) normalizing measurement unit(s) across multiple data sources to a common scale (e.g., converting screen time from different sources into total daily sedentary minutes, etc.), (ii) aligning time-series data for consistency across sampling intervals (e.g., synchronizing media viewing logs with wearable activity data in 1-minute intervals, etc.), and/or (iii) mapping unstructured data into a predefined data schema (e.g., extracting workout-related keywords from social media posts and tagging them under ‘physical activity behavior’, etc.).

In block 305, the analysis platform 103 may input the transformed data into the trained machine-learning model to identify patterns within the data indicating deviation from the predefined activity threshold(s). In one instance, the predefined activity threshold(s) may represent baseline healthy behaviors, such as steps taken per hour, average daily screen time, or frequency of physical activity, and may be calibrated to individual user profiles based upon various criteria (e.g., age, goals, medical backgrounds, etc.). The trained machine-learning model may continuously analyze the transformed data, and may detect deviation from the predefined activity threshold(s) (e.g., extended sedentary behaviors, reduced engagement in wellness routine, prolonged inactivity without scheduled breaks, or missed goals). The trained machine-learning model may flag the deviation and log the context, which may trigger context-sensitive prompts or reminders encouraging the user to take corrective actions. Over time, the system may refine its threshold sensitivity and notifications based upon individual response patterns, helping the user maintain adherence to health objectives in a data-driven manner.

In block 307, the analysis platform 103 may determine a correlation between the media content displayed in a device associated with the user (e.g., user device 101) and the deviation from the predefined activity threshold(s). In one instance, the analysis platform 103 may compare media consumption data and corresponding user activity data to identify pattern(s) indicative of (i) a period of inactivity, (ii) reduced task completion, (iii) increased sedentary duration, (iv) delayed initiation of physical tasks, or (v) skipped scheduled routine. In one example, the analysis platform 103 may analyze timestamps associated with both user activity data and media consumption logs to identify whether a specific type of content (e.g., long-form television shows, binge-watched series) is frequently linked to periods of extended inactivity. The analysis platform 103 may use this correlation to build behavioral context, recognizing patterns where certain media formats or genres are statistically associated with a drop in physical engagement.

In block 309, the analysis platform 103 may select alternative content data for replacing or supplementing the media content displayed in the device based upon the determined correlation. The alternative content data may be selected based upon historical data indicating effectiveness of similar content types in improving behavior of the user in (i) reducing prolonged inactivity, (ii) increasing task completion rates, (iii) reducing sedentary duration, and/or (iv) initiating physical tasks or scheduled routine.

In block 311, the analysis platform 103 may modify the media content displayed in the device to include the alternative content data. The analysis platform 103 may superimpose the alternative content data in real-time over the media content displayed in the device based upon the deviation from the predefined activity threshold(s). In one instance, the analysis platform 103 may determine that the user consistently remains inactive while watching late-night comedy shows and may identify a correlation between this media and deviation from the user’s predefined activity threshold. In response, the analysis platform 103 may modify the media content displayed upon the smart television by inserting short wellness programs (e.g., guided stretches or posture reminders) between episodes or during natural pause points.

The analysis platform 103 may monitor the media content displayed in the device for interval(s). The analysis platform 103 may generate a display of one or more responses during the interval(s) based upon the deviation from the predefined activity threshold(s). In one instance, during commercial breaks, the analysis platform 103 may replace standard advertisements with short clips promoting light physical activities or mental wellness tips. In another instance, the analysis platform 103 may track task(s) associated with the user. The analysis platform 103 may identify at least one overdue task from the task(s) based upon a predefined completion timeline. The analysis platform 103 may generate a display of one or more responses during the interval(s) to encourage completion of the overdue task.

The analysis platform 103 may monitor adherence to the one or more responses by the user based upon (i) completion rate(s) (e.g., percentage of suggested physical activities completed after a prompt, etc.), (ii) user engagement data (e.g., time spent viewing or interacting with recommended wellness content, etc.), and/or (iii) interaction pattern(s) (e.g., frequency and timing of user responses to prompts, etc.). The analysis platform 103 may determine progress of the user towards the predefined activity threshold(s) (e.g., completion of 8,000 of the 10,000 daily steps) or completion of an overdue task (e.g., finishing a scheduled 5-minute stretching session that was missed earlier).

In one instance, the analysis platform 103 may adjust the responses based upon the determined progress. For example, if the user is nearing an activity threshold, having completed 8,000 out of 10,000 steps, the analysis platform 103 may generate encouraging messages or suggest a short walk to help close the gap. Conversely, if little progress has been made or an overdue task remains incomplete, the analysis platform 103 may issue frequent prompts, such as sending a notification with simplified actions (e.g., stand up and stretch for 2 minutes). Over time, the analysis platform 103 may learn which type of intervention yields the best results for each user and may dynamically adapt the tone, frequency, and delivery method of these responses.

### Exemplary Interactive Wellness Media Environment

FIG. 3B depicts a diagram illustrating a system for analyzing user behavior and media consumption patterns for generating personalized recommendations, according to certain aspects of the disclosure. In a connected media environment 302, a user 304 may be watching content upon a smart television while holding a mobile phone. The user 304 is wearing a smartwatch and smart clothing embedded with a plurality of sensors. These devices may continuously communicate with the analysis platform 103 configured to promote health and wellness behaviors. The analysis platform 103 may aggregate, synchronize, and/or process multimodal data streams in real-time to monitor the user’s physiological state, behavioral patterns, and/or media consumption context.

As the user watches the smart television, the analysis platform 103 may detect opportunities for health interventions based upon both user behaviors and the nature of the content being consumed. The analysis platform 103 may identify a window for influence, such as the onset of a commercial break, and may dynamically adjust the content upon the smart television. Specifically, the screen may display curated media 306 promoting physical exercise, healthy dietary habits, and/or stress-reduction practices (e.g., guided breathing or mindfulness exercises). These interventions may be selected utilizing the trained machine-learning model that may tailor the content to the user’s historical preferences, biometric indicators, and/or predefined wellness goals.

During commercial breaks, the analysis platform 103 may coordinate data inputs from the smartwatch, smart clothing, and/or mobile phone to confirm user inactivity. Upon verifying sedentary posture or lack of movement, the analysis platform 103 may transmit real-time prompts via visual and haptic cues across devices, suggesting simple, achievable activities, such as standing, stretching, and/or walking. These prompts may be calibrated using sensor fusion techniques that evaluate motion, heart rate, and/or posture, ensuring recommendations are contextually appropriate and achievable within the user’s current physical state and environment.

The analysis platform 103 may further enforce behavioral rules by regulating access to passive media content. For example, the analysis platform 103 may implement a monitoring mechanism where continuation of a video stream may be conditioned upon successful completion of a prompted activity. Such rule-based interventions may operate upon a reinforcement learning framework that maximizes user compliance while minimizing disruptions. By associating healthy behaviors with positive reinforcements, like media continuation or gamified rewards, the analysis platform 103 may foster habit formation and long-term behavioral change.

Additionally, the analysis platform 103 may track personal productivity indicators and health tasks, such as overdue wellness activities or uncompleted daily goals. By analyzing data from various productivity applications and/or user-entered data, the analysis platform 103 may identify tasks that remain incomplete and select commercial break intervals to deliver gentle reminders. These notifications may be designed with minimal cognitive load, using adaptive timing and multi-device delivery to maximize user receptivity without causing notification fatigue.

This integrated approach may enhance user well-being by embedding proactive health interventions into natural entertainment patterns. Through continuous monitoring and adaptive feedback, the analysis platform 103 may support behavior change without relying upon drastic lifestyle shifts. The analysis platform 103 may transform traditionally passive screen time into an active and productive component.

### Exemplary Driving Behavior Analysis and Assistance System

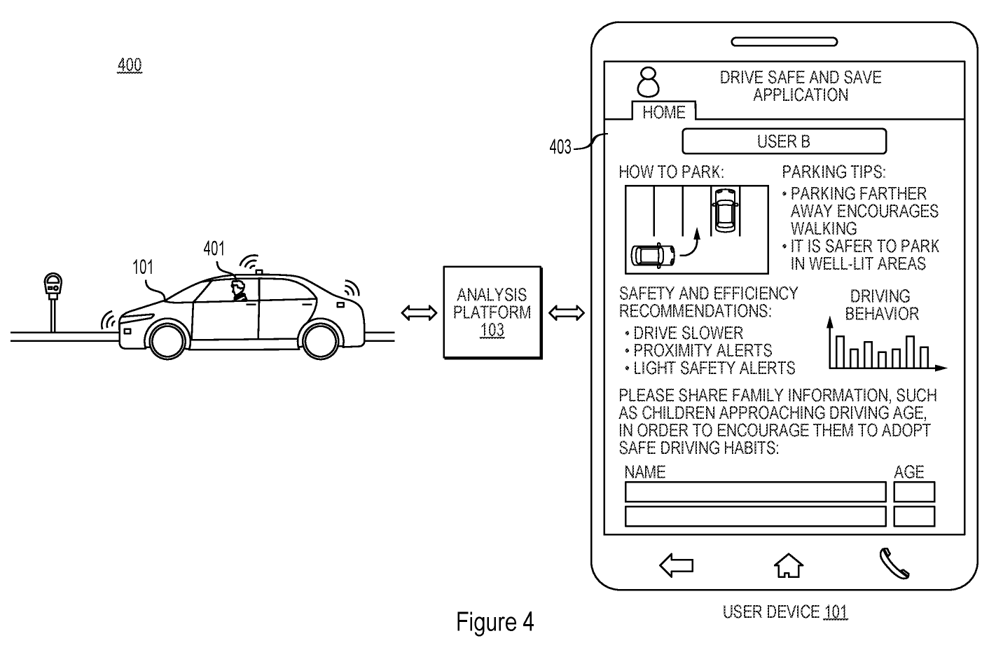

FIG. 4 depicts a diagram illustrating a system for analyzing real-time driving and environmental data for generating personalized recommendations, according to certain aspects of the disclosure. The system 400 may leverage smart vehicle sensors, geolocation data, and/or environmental context to deliver personalized driving and/or parking recommendations to user 401. In one scenario, the user 401 may be driving a smart vehicle upon a congested road, and the system 400 may initiate communication between the smart vehicle and the analysis platform 103. The analysis platform 103 may aggregate and process sensor data, such as speed, braking patterns, traffic density, and/or available parking spaces to determine optimal parking strategies.

Upon detecting that the user 401 is attempting to park, the analysis platform 103 may process real-time data and perform predictive modeling to generate personalized parking recommendations. The analysis platform 103 may evaluate the current location, lighting conditions, pedestrian density, proximity to the user’s destination, and/or walking routes to suggest optimal parking spots. In one example, the analysis platform 103 may recommend a farther parking space to encourage physical activity or advise selecting a parking area with enhanced lighting to improve safety. These recommendations may be rendered through the in-vehicle display or companion mobile application (e.g., user interface 403), which may feature an AI-driven avatar that may provide clear visual and voice instructions upon how to park (e.g., angle or parallel parking guidance) and/or where to park (e.g., specific lot or street segment).

In one instance, the AI-driven avatar may serve as an intelligent interface that may interpret the system’s output and communicate intuitive guidance to the user 401. Using real-time telemetry from vehicle-mounted cameras, lidar, and/or proximity sensors, the AI-driven avatar may overlay augmented-reality indicators such as arrows, parking zone highlights, and/or alignment lines into the vehicle’s heads-up display or mobile application screen (e.g., user interface 403). This may facilitate seamless, user-friendly navigation into available parking spots, reducing the cognitive load to the driver and may minimize parking errors or potential collision with surrounding objects.

In addition to parking assistance, the analysis platform 103 may continuously monitor the driving behavior of the user 401 to promote safety and efficiency. The analysis platform 103 may issue context-sensitive alerts based upon detected driving patterns, such as recommending reduced speed during sudden acceleration events, providing forward collision warnings when the following distance may be inadequate, and/or issuing lane departure alerts if the vehicle drifts without signaling. Environmental data, such as visibility or weather conditions, may be considered to offer lighting and/or safety-related feedback, enhancing situational awareness during driving sessions.

In one instance, the analysis platform 103 may incentivize proactive driving behavior and family preparation. The user 401 may be rewarded for voluntarily sharing contextual family information, such as details regarding children nearing driving age. This information may help the analysis platform 103 to tailor recommendations related to driver education, safe vehicle operation, and/or defensive driving practices, which may be critical in preparing future drivers. The analysis platform 103 may also prompt the user 401 to participate in driving simulations that may be shared with the child, further promoting safety in multi-generational driving households.

The described system 400 may deliver several benefits, including increased user safety, improved parking efficiency, and/or support for long-term wellness objectives through behavioral nudges (e.g., encouraging walking or exercise). By incorporating real-time analytics, intelligent feedback, and/or a user-centric AI avatar, the analysis platform 103 may transform the in-vehicle experience into a proactive, personalized assistant.

### Exemplary Machine-Learning Techniques

One or more implementations disclosed herein may include and/or may be implemented using a machine-learning model. For example, one or more of the modules of the analysis platform 103 may be implemented using a machine-learning model and/or may be used to train the machine-learning model. A given machine-learning model may be trained using the data flow 500 of FIG. 5. Training data 512 may include one or more of stage inputs 514 and known outcomes 518 related to the machine-learning model to be trained. The stage inputs 514 may be from any applicable source including text, visual representations, data, values, comparisons, stage outputs, e.g., one or more outputs from one or more actions or operations from FIGS. 2A-4. The known outcomes 518 may be included for the machine-learning models generated based upon supervised or semi-supervised training. An unsupervised machine-learning model may not be trained using known outcomes 518. Known outcomes 518 may include known or desired outputs for future inputs similar to, or in the same category as, stage inputs 514 that do not have corresponding known outputs.

The training data 512 and a training algorithm 520, e.g., one or more of the modules implemented using the machine-learning model and/or may be used to train the machine-learning model, may be provided to a training component 530 that may apply the training data 512 to the training algorithm 520 to generate the machine-learning model. According to an implementation, the training component 530 may be provided comparison results 516 that compare a previous output of the corresponding machine-learning model to apply the previous result to re-train the machine-learning model. The comparison results 516 may be used by training component 530 to update the corresponding machine-learning model. The training algorithm 520 may utilize machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, models specifically discussed in the present disclosure, or the like.

The machine-learning model used herein may be trained and/or used by adjusting one or more weights and/or one or more layers of the machine-learning model. For example, during training, a given weight may be adjusted (e.g., increased, decreased, removed) based upon training data or input data. Similarly, a layer may be updated, added, or removed based upon training data/and or input data. The resulting outputs may be adjusted based upon the adjusted weights and/or layers.

In general, any process or operation discussed in this disclosure is understood to be computer-implementable, such as the processes illustrated in FIGS. 1-2B may be performed by one or more processors of a computer system as described herein. A process or process action or operation performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

### Exemplary Computing Environment

In general, any process or operation discussed in this disclosure is understood to be computer-implementable, such as the processes illustrated in FIGS. 1-4 and may be performed by one or more processors of a computer system as described herein. A process or process action or operation performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

FIG. 6 illustrates an implementation of a computer system that may execute techniques presented herein. The computer system 600 may include a set of instructions that can be executed to cause the computer system 600 to perform any one or more of the methods or computer based functions disclosed herein. The computer system 600 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, “analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In a networked deployment, the computer system 600 may operate in the capacity of a server or as a client user computer in a server-client user network environment, or as a peer computer system in a peer-to-peer (or distributed) network environment. The computer system 600 can also be implemented as or incorporated into various devices, such as a personal computer (PC), a tablet PC, a Personal Digital Assistant (PDA), a mobile device, a palmtop computer, a laptop computer, a desktop computer, a web appliance, or any other machine capable of executing a set of instructions (sequential or otherwise) that specify actions to be taken by that machine. In a particular implementation, the computer system 600 may be implemented using electronic devices that provide voice, video, or data communication. Further, while the computer system 600 is illustrated as a single system, the term “system” shall also be taken to include any collection of systems or sub-systems that individually or jointly execute a set, or multiple sets, of instructions to perform one or more computer functions.

As illustrated in FIG. 6, the computer system 600 may include a processor 602, e.g., a Central Processing Unit (CPU), a Graphics Processing Unit (GPU), or both. The processor 602 may be a component in a variety of systems. For example, the processor 602 may be part of a standard personal computer or a workstation. The processor 602 may be one or more processors, digital signal processors, application specific integrated circuits, field programmable gate arrays, servers, networks, digital circuits, analog circuits, combinations thereof, or other now known or later developed devices for analyzing and processing data. The processor 602 may implement a software program, such as code generated manually (i.e.,programmed).

The computer system 600 may include a memory 604 that can communicate via bus 608. The memory 604 may be a main memory, a static memory, or a dynamic memory. The memory 604 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 604 may include a cache or random-access memory for the processor 602. In alternative implementations, the memory 604 is separate from the processor 602, such as a cache memory of a processor, the system memory, or other memory.

The memory 604 may be an external storage device or database for storing data. Examples may include a hard drive, compact disc (“CD”), digital video disc (“DVD”), memory card, memory stick, floppy disc, universal serial bus (“USB”) memory device, or any other device operative to store data. The memory 604 is operable to store instructions executable by the processor 602. The functions, acts or tasks illustrated in the figures or described herein may be performed by the processor 602 executing the instructions stored in the memory 604. The functions, acts, or tasks are independent of the particular type of instruction set, storage media, processor or processing strategy and may be performed by software, hardware, integrated circuits, firmware, micro-code and the like, operating alone or in combination. Likewise, processing strategies may include multiprocessing, multitasking, parallel processing, and the like.

As shown, the computer system 600 may further include a display 610, such as a liquid crystal display (LCD), an organic light emitting diode (OLED), a flat panel display, a solid-state display, a cathode ray tube (CRT), a projector, a printer or other now known or later developed display device for outputting determined information. The display 610 may act as an interface for the user to see the functioning of the processor 602, or specifically as an interface with the software stored in the memory 604 or in the drive unit 606.

Additionally or alternatively, the computer system 600 may include an input/output device 612 configured to allow a user to interact with any of the components of the computer system 600. The input/output device 612 may be a number pad, a keyboard, or a cursor control device, such as a mouse, or a joystick, touch screen display, remote control, or any other device operative to interact with the computer system 600.

The computer system 600 may also or alternatively include drive unit 606 implemented as a disk or optical drive. The drive unit 606 may include a computer-readable medium 622 in which one or more sets of instructions 624, e.g., software, can be embedded. Further, instructions 624 may embody one or more of the methods or logic as described herein. The instructions 624 may reside completely or partially within the memory 604 and/or within the processor 602 during execution by the computer system 600. The memory 604 and the processor 602 also may include computer-readable media as discussed above.

In some systems, computer-readable medium 622 may include the set of instructions 624 or receive and execute the set of instructions 624 responsive to a propagated signal so that a device connected to network 630 can communicate voice, video, audio, images, or any other data over the network 630. Further, the set of instructions 624 may be transmitted or received over the network 630 via communication port or interface 620, and/or using bus 608. The communication port or interface 620 may be a part of the processor 602 or may be a separate component. The communication port or interface 620 may be created in software or may be a physical connection in hardware.

The communication port or interface 620 may be configured to connect with a network 630, external media, the display 610, or any other components in computer system 600, or combinations thereof. The connection with the network 630 may be a physical connection, such as a wired Ethernet connection or may be established wirelessly as discussed below. Likewise, the additional connections with other components of the computer system 600 may be physical connections or may be established wirelessly. The network 630 may alternatively be directly connected to the bus 608.

While the computer-readable medium 622 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that causes a computer system to perform any one or more of the methods or operations disclosed herein. The computer-readable medium 622 may be non-transitory, and may be tangible.

The computer-readable medium 622 may include a solid-state memory such as a memory card or other package that houses one or more non-volatile read-only memories. The computer-readable medium 622 can be a random-access memory or other volatile re-writable memory. Additionally or alternatively, the computer-readable medium 622 may include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations may broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

Computer system 600 may be connected to network 630. The network 630 may define one or more networks including wired or wireless networks. The wireless network may be a cellular telephone network, an 802.10, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 630 may include wide area networks (WAN), such as the Internet, local area networks (LAN), campus area networks, metropolitan area networks, a direct connection such as through a Universal Serial Bus (USB) port, or any other networks that may allow for data communication.

The network 630 may be configured to couple one computing device to another computing device to enable communication of data between the devices. The network 630 may generally be enabled to employ any form of machine-readable media for communicating information from one device to another. The network 630 may include communication methods by which information may travel between computing devices.

The network 630 may be divided into sub-networks. The sub-networks may allow access to all of the other components connected thereto or the sub-networks may restrict access between the components. The network 630 may be regarded as a public or private network connection and may include, for example, a virtual private network or an encryption or other security mechanism employed over the public Internet, or the like.

### Exemplary Embodiment

A computer-implemented method for processing data to generate one or more context-aware adaptive responses may be provided. The computer-implemented method may be performed by one or more local or remote processors of a computing system in communication with one or more local or remote data sources. The computer-implemented method may include (1) receiving, by the one or more processors, data associated with at least one user from one or more sensors; (2) processing, by the one or more processors, the received data to transform the received data into a structured format; (3) inputting, by the one or more processors, the processed data into a trained machine-learning model for generating the one or more context-aware adaptive responses for the at least one user; (4) generating, by the one or more processors, a display of the one or more context-aware adaptive responses and a corresponding avatar; (5) outputting, by the one or more processors, the display (such as visual, textual, graphical, audible, verbal, and/or other output) and the avatar to a user interface of at least one device associated with the at least one user; (6) in response to the outputting, monitoring, by the one or more processors, one or more activities performed by the at least one user based upon the display of the one or more context-aware adaptive responses; and/or (7) updating, by the one or more processors, the avatar based upon the monitored one or more user activities.

In some embodiments, the voice bots or chatbots may be configured to utilize AI and/or ML techniques, such as for input or output devices. For instance, a voice bot or chatbot may be a ChatGPT chatbot, an InstructGPT bot, a Codex bot, or a Google Bard bot. The voice bot or chatbot may employ supervised or unsupervised ML techniques, which may be followed by, and/or used in conjunction with, reinforced or reinforcement learning techniques. The voice bot or chatbot may employ the techniques utilized for ChatGPT, InstructGPT bot, Codex bot, or Google Bard bot.

In certain aspects, the trained machine-learning model may generate the one or more context-aware adaptive responses by (a) analyzing, by the one or more processors, utilizing the trained machine-learning model, the processed data to extract one or more statistical features, one or more temporal features, and/or one or more contextual features; (b) identifying, by the one or more processors, utilizing the trained machine-learning model, one or more patterns corresponding to the extracted one or more features. The one or more patterns may indicate (i) one or more behavioral profiles, (ii) one or more temporal health state transitions, and/or (iii) one or more deviations from normative lifestyle baselines; (c) generating, by the one or more processors, utilizing the trained machine-learning model, one or more predictions based upon the one or more patterns. The one or more predictions may include (i) one or more dynamically inferred user states, (ii) an estimated progression of behavioral trajectories, and/or (iii) a probability score for possible interventions; and/or (d) selecting, by the one or more processors, utilizing the trained machine-learning model, one or more of the one or more context-aware adaptive responses based upon the one or more predictions.

Additionally or alternatively, the one or more context-aware adaptive responses may include (i) an activity suggestion, (ii) one or more personalized health plans, (iii) one or more stress management techniques, (iv) one or more sleep optimization strategies, (v) one or more substance use reduction plans, and/or (vi) one or more preventive healthcare screenings.

In various embodiments, monitoring the one or more activities performed by the at least one user may include (a) receiving, by the one or more processors, one or more real-time data streams from the one or more sensors; and/or (b) analyzing, by the one or more processors, the one or more real-time data streams for determining a level of compliance corresponding to the one or more context-aware adaptive responses using a rule-based analysis. The rule-based analysis may include one or more predefined thresholds for the one or more activities to assess the level of compliance.

In certain embodiments, updating the avatar based upon the monitored one or more user activities may include adjusting, by the one or more processors, an appearance and one or more characteristics of the avatar based upon a condition improvement or a condition deterioration corresponding to the at least one user. The appearance and the one or more characteristics may reflect a success or a failure in completing the one or more context-aware adaptive responses.

In some embodiments, updating the avatar may include modifying, by the one or more processors, one or more facial expressions, a posture, or a clothing item of the avatar to visually indicate the success or the failure. The success may be represented by one or more positive traits of the avatar including (i) an upright posture, (ii) one or more happy facial expressions, and/or (iii) an athletic clothing item in one or more bright colors. The failure may be represented by one or more negative traits of the avatar including (i) a slouched posture, (ii) one or more sad facial expressions, and/or (iii) an untidy clothing item in one or more dark colors.

Additionally or alternatively, the data associated with the at least one user may include (i) behavioral data, (ii) physiological data, (iii) contextual data, (iv) historical data, and/or (v) health data.

In certain aspects, the one or more sensors may include (i) a motion sensor for detecting user movement and physical activity, (ii) a heart rate sensor for detecting pulse and heart rate variability, (iii) a sleep monitoring sensor for determining one or more sleep patterns and a duration, (iv) a temperature sensor for detecting one or more changes in body temperature, (v) a microphone for detecting one or more sounds associated with user behavior, (vi) a geolocation sensor for determining a spatial movement pattern and a location context, and/or (vii) a pressure sensor embedded in one or more smart clothing items for monitoring a posture and a respiration.

In various embodiments, transforming the received data into the structured format may include (i) normalizing one or more measurement units across multiple data sources to a common scale, (ii) aligning time-series data for consistency across one or more sampling intervals, and/or (iii) mapping unstructured data into a predefined data schema.

In certain embodiments, generating, by the one or more processors, one or more notifications in the user interface. The one or more notifications may include (i) one or more rewards, (ii) one or more premium discounts, (iii) one or more achievement-based badges, (iv) one or more goal-setting challenges, and/or (v) enhancing the avatar to reflect user progress.

A computer system for processing data to generate one or more context-aware adaptive responses may be provided. The computer system may include one or more processors of a computing system, and at least one non-transitory computer readable medium storing instructions which, when executed by the one or more processors, cause the one or more processors to perform operations. The computer system may perform operations including (1) receiving data associated with at least one user from one or more sensors; (2) processing the received data to transform the received data into a structured format; (3) inputting the processed data into a trained machine-learning model for generating the one or more context-aware adaptive responses for the at least one user; (4) generating a display of the one or more context-aware adaptive responses and a corresponding avatar; (5) outputting the display and the avatar to a user interface of at least one device associated with the at least one user; (6) in response to the outputting, monitoring one or more activities performed by the at least one user based upon the display of the one or more context-aware adaptive responses; and/or (7) updating the avatar based upon the monitored one or more user activities.

In certain aspects, the trained machine-learning model may generate the one or more context-aware adaptive responses by (a) analyzing, utilizing the trained machine-learning model, the processed data to extract one or more statistical features, one or more temporal features, or one or more contextual features; (b) identifying, utilizing the trained machine-learning model, one or more patterns corresponding to the extracted one or more features. The one or more patterns may indicate (i) one or more behavioral profiles, (ii) one or more temporal health state transitions, and/or (iii) one or more deviations from normative lifestyle baselines; (c) generating, utilizing the trained machine-learning model, one or more predictions based upon the one or more patterns. The one or more predictions may include (i) one or more dynamically inferred user states, (ii) an estimated progression of behavioral trajectories, and/or (iii) a probability score for possible interventions; and/or (d) selecting, utilizing the trained machine-learning model, one or more of the one or more context-aware adaptive responses based upon the one or more predictions.

Additionally or alternatively, the one or more context-aware adaptive responses may include (i) an activity suggestion, (ii) one or more personalized health plans, (iii) one or more stress management techniques, (iv) one or more sleep optimization strategies, (v) one or more substance use reduction plans, and/or (vi) one or more preventive healthcare screenings.

In various embodiments, monitoring the one or more activities performed by the at least one user may include (a) receiving one or more real-time data streams from the one or more sensors; and/or (b) analyzing the one or more real-time data streams for determining a level of compliance corresponding to the one or more context-aware adaptive responses using a rule-based analysis. The rule-based analysis may include one or more predefined thresholds for the one or more activities to assess the level of compliance.

In certain embodiments, updating the avatar based upon the monitored one or more user activities may include adjusting an appearance and one or more characteristics of the avatar based upon a condition improvement or a condition deterioration corresponding to the at least one user. The appearance and the one or more characteristics may reflect a success or a failure in completing the one or more context-aware adaptive responses.

In some embodiments, updating the avatar may include (a) modifying one or more facial expressions, a posture, or a clothing item of the avatar to visually indicate the success or the failure. The success may be represented by one or more positive traits of the avatar including (i) an upright posture, (ii) one or more happy facial expressions, and/or (iii) an athletic clothing item in one or more bright colors. The failure may be represented by one or more negative traits of the avatar including (i) a slouched posture, (ii) one or more sad facial expressions, and/or (iii) an untidy clothing item in one or more dark colors.

Additionally or alternatively, the data associated with the at least one user may include (i) behavioral data, (ii) physiological data, (iii) contextual data, (iv) historical data, or (v) health data.

A non-transitory computer readable medium for processing data to generate one or more context-aware adaptive responses may be provided. The non-transitory computer readable medium may store instructions which, when executed by one or more processors, cause the one or more processors to perform operations. The one or more processors may perform operations including (1) receiving data associated with at least one user from one or more sensors; (2) processing the received data to transform the received data into a structured format; (3) inputting the processed data into a trained machine-learning model for generating the one or more context-aware adaptive responses for the at least one user; (4) generating a display of the one or more context-aware adaptive responses and a corresponding avatar; (5) outputting the display and the avatar to a user interface of at least one device associated with the at least one user; (6) in response to the outputting, monitoring one or more activities performed by the at least one user based upon the display of the one or more context-aware adaptive responses; and/or (7) updating the avatar based upon the monitored one or more user activities.

In certain aspects, the trained machine-learning model may generate the one or more context-aware adaptive responses by (a) analyzing utilizing the trained machine-learning model, the processed data to extract one or more statistical features, one or more temporal features, or one or more contextual features; (b) identifying utilizing the trained machine-learning model, one or more patterns corresponding to the extracted one or more features. The one or more patterns may indicate (i) one or more behavioral profiles, (ii) one or more temporal health state transitions, and/or (iii) one or more deviations from normative lifestyle baselines; (c) generating utilizing the trained machine-learning model, one or more predictions based upon the one or more patterns. The one or more predictions may include (i) one or more dynamically inferred user states, (ii) an estimated progression of behavioral trajectories, or (iii) a probability score for possible interventions; and/or (d) selecting utilizing the trained machine-learning model, one or more of the one or more context-aware adaptive responses based upon the one or more predictions.

In various embodiments, monitoring the one or more activities performed by the at least one user may include (a) receiving one or more real-time data streams from the one or more sensors; and/or (b) analyzing the one or more real-time data streams for determining a level of compliance corresponding to the one or more context-aware adaptive responses using a rule-based analysis. The rule-based analysis may include one or more predefined thresholds for the one or more activities to assess the level of compliance.

A computer-implemented method for dynamic integration of sensor data utilizing a trained machine-learning model for modifying media content may be provided. The computer-implemented method may be performed by one or more local or remote processors of a computing system in communication with one or more local or remote data sources. The computer-implemented method may include (1) receiving, by the one or more processors, data associated with at least one user from one or more sensors; (2) transforming, by the one or more processors, the received data into a structured format; (3) inputting, by the one or more processors, the transformed data into the trained machine-learning model to identify one or more patterns within the data indicating a deviation from one or more predefined activity thresholds; (4) determining, by the one or more processors, a correlation between the media content displayed in at least one device and the deviation from the one or more predefined activity thresholds; (5) based upon the determined correlation, selecting, by the one or more processors, alternative content data for replacing or supplementing the media content displayed in the at least one device; and/or (6) modifying, by the one or more processors, the media content displayed in the at least one device to include the alternative content data.

In certain aspects, modifying the media content may include (a) monitoring, by the one or more processors, the media content displayed in the at least one device for one or more intervals, and /or (b) generating, by the one or more processors, a display of one or more responses during the one or more intervals based upon the deviation from the one or more predefined activity thresholds.

In certain embodiments, generating the display of the one or more responses may include (a) tracking, by the one or more processors, one or more tasks associated with the at least one user; (b) identifying, by the one or more processors, at least one overdue task from the one or more tasks based upon a predefined completion timeline; and/or (c) generating, by the one or more processors, the display of the one or more responses during the one or more intervals to encourage task completion.

In some embodiments, generating the display of the one or more responses may further include (a) monitoring, by the one or more processors, adherence to the one or more responses by the at least one user based upon (i) one or more completion rates, (ii) user engagement data, and/or (iii) one or more interaction patterns; (b) determining, by the one or more processors, progress of the at least one user towards the one or more predefined activity thresholds or completion of the at least one overdue task; and/or adjusting, by the one or more processors, the one or more responses based upon the determined progress.

In various embodiments, determining the correlation between the media content and the deviation from the one or more predefined activity thresholds may include comparing, by the one or more processors, media consumption data and corresponding user activity data to identify one or more patterns indicative of (i) a period of inactivity, (ii) a reduced task completion, (iii) an increased sedentary duration, (iv) a delayed initiation of one or more physical tasks, and/or (v) a skipped scheduled routine.

Additionally or alternatively, the alternative content data may be selected based upon historical data indicating effectiveness of one or more similar content types in improving behavior of the at least one user in (i) reducing prolonged inactivity, (ii) increasing one or more task completion rates, (iii) reducing a sedentary duration, and/or (iv) initiating one or more physical tasks or a scheduled routine.

In certain aspects, modifying the media content displayed in the at least one device with the alternative content data may include superimposing, by the one or more processors, the alternative content data in real-time over the media content displayed in the at least one device based upon the deviation from the one or more predefined activity thresholds.

In various embodiments, the data associated with the at least one user may include (i) activity tracking data, (ii) media consumption data, (iii) one or more task completion records, (iv) one or more predetermined health related goals, and/or (v) historical adherence data to the one or more predefined activity thresholds.

Additionally or alternatively, the one or more sensors may include (i) a motion sensor for detecting user movement and physical activity, (ii) a heart rate sensor for monitoring cardiovascular activity, (iii) a pressure sensor embedded in smart clothing for monitoring posture and respiration, (iv) a camera sensor for detecting facial expressions or eye tracking for attention monitoring, and/or (v) a screen usage detection sensor for tracking one or more media consumptions patterns.

In certain embodiments, transforming the received data into the structured format may include (i) normalizing one or more measurement units across the one or more data sources to a common scale, (ii) aligning time-series data for consistency across one or more sampling intervals, and/or (iii) mapping unstructured data into a predefined data schema.

A computer system for dynamic integration of sensor data utilizing a trained machine-learning model for modifying media content may be provided. The computer system may include one or more processors of a computing system, and at least one non-transitory computer readable medium storing instructions which, when executed by the one or more processors, cause the one or more processors to perform operations. The computer system may perform operations including (1) receiving data associated with at least one user from one or more sensors; (2) transforming the received data into a structured format; (3) inputting the transformed data into the trained machine-learning model to identify one or more patterns within the data indicating a deviation from one or more predefined activity thresholds; (4) determining a correlation between the media content displayed in at least one device and the deviation from the one or more predefined activity thresholds; (5) based upon the determined correlation, selecting alternative content data for replacing or supplementing the media content displayed in the at least one device; and/or (6) modifying the media content displayed in the at least one device to include the alternative content data.

In certain aspects, modifying the media content may include (a) monitoring the media content displayed in the at least one device for one or more intervals, and/or (b) generating a display of one or more responses during the one or more intervals based upon the deviation from the one or more predefined activity thresholds.

In certain embodiments, generating the display of the one or more responses may include (a) tracking one or more tasks associated with the at least one user; (b) identifying at least one overdue task from the one or more tasks based upon a predefined completion timeline; and/or (c) generating the display of the one or more responses during the one or more intervals to encourage task completion.

In some embodiments, generating the display of the one or more responses may further include (a) monitoring adherence to the one or more responses by the at least one user based upon (i) one or more completion rates, (ii) user engagement data, or (iii) one or more interaction patterns; (b) determining progress of the at least one user towards the one or more predefined activity thresholds or completion of the at least one overdue task; and/or (c) adjusting the one or more responses based upon the determined progress.

In various embodiments, determining the correlation between the media content and the deviation from the one or more predefined activity thresholds may include comparing media consumption data and corresponding user activity data to identify one or more patterns indicative of (i) a period of inactivity, (ii) a reduced task completion, (iii) an increased sedentary duration, (iv) a delayed initiation of one or more physical tasks, or (v) a skipped scheduled routine.

Additionally or alternatively, the alternative content data may be selected based upon historical data indicating effectiveness of one or more similar content types in improving behavior of the at least one user in (i) reducing prolonged inactivity, (ii) increasing one or more task completion rates, (iii) reducing a sedentary duration, and/or (iv) initiating one or more physical tasks or a scheduled routine.

In certain aspects, modifying the media content displayed in the at least one device with the alternative content data may include superimposing the alternative content data in real-time over the media content displayed in the at least one device based upon the deviation from the one or more predefined activity thresholds.

A non-transitory computer readable medium for dynamic integration of sensor data utilizing a trained machine-learning model for modifying media content may be provided. The non-transitory computer readable medium may store instructions which, when executed by one or more processors, cause the one or more processors to perform operations. The one or more processors may perform operations including (1) receiving data associated with at least one user from one or more sensors; (2) transforming the received data into a structured format; (3) inputting the transformed data into the trained machine-learning model to identify one or more patterns within the data indicating a deviation from one or more predefined activity thresholds; (4) determining a correlation between the media content displayed in at least one device and the deviation from the one or more predefined activity thresholds;(5) based upon the determined correlation, selecting alternative content data for replacing or supplementing the media content displayed in the at least one device; and/or (6) modifying the media content displayed in the at least one device to include the alternative content data.

In certain aspects, modifying the media content may include (a) monitoring the media content displayed in the at least one device for one or more intervals, and/or (b) generating a display of one or more responses during the one or more intervals based upon the deviation from the one or more predefined activity thresholds.

In certain embodiments, generating the display of the one or more responses may include (a) tracking one or more tasks associated with the at least one user; (b) identifying at least one overdue task from the one or more tasks based upon a predefined completion timeline; and/or (c) generating the display of the one or more responses during the one or more intervals to encourage task completion.

### Additional Considerations

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the actions, operations, and/or functionality of computer-implemented methods discussed are performed in one embodiment by an appropriate processor (or processors) of a processing (i.e.,computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

The present embodiments envision collecting and analyzing user or customer data, such as sensor data, mobile device data, smart or autonomous data, smart appliance data, etc. associated with the user or customer devices or computing devices. Such data may be collected and analyzed once the user or customer affirmatively opts into a program, such as a customer loyalty or rewards program, or the customer otherwise affirmatively consents to data being generated by their devices being utilized, collected, and/or analyzed.

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘_______’ is hereby defined to mean…” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Finally, unless a claim element is defined by expressly reciting the word “means” and a function without the recital of any structure, it is not intended that the scope of any claim element be interpreted based upon the application of 35 U.S.C. § 112(f).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in exemplary configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied upon a non-transitory, tangible machine-readable medium) or hardware. In hardware, the routines, etc., are tangible units capable of performing certain operations and may be configured or arranged in a certain manner. In exemplary embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate upon a resource (e.g., a collection of information).

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some exemplary embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

What is claimed is:

1. A computer-implemented method for processing data to generate one or more context-aware adaptive responses, the computer-implemented method performed by one or more processors of a computing system in communication with one or more data sources, the computer-implemented method comprising:
receiving, by the one or more processors, data associated with at least one user from one or more sensors;
processing, by the one or more processors, the received data to transform the received data into a structured format;
inputting, by the one or more processors, the processed data into a trained machine-learning model for generating the one or more context-aware adaptive responses for the at least one user;
generating, by the one or more processors, a display of the one or more context-aware adaptive responses and a corresponding avatar;
outputting, by the one or more processors, the display and the avatar to a user interface of at least one device associated with the at least one user;
in response to the outputting, monitoring, by the one or more processors, one or more activities performed by the at least one user based upon the display of the one or more context-aware adaptive responses; and
updating, by the one or more processors, the avatar based upon the monitored one or more user activities.

2. The computer-implemented method of claim 1, wherein the trained machine-learning model generating the one or more context-aware adaptive responses, further comprises:
analyzing, by the one or more processors, utilizing the trained machine-learning model, the processed data to extract one or more statistical features, one or more temporal features, or one or more contextual features;
identifying, by the one or more processors, utilizing the trained machine-learning model, one or more patterns corresponding to the extracted one or more features, wherein the one or more patterns indicate (i) one or more behavioral profiles, (ii) one or more temporal health state transitions, or (iii) one or more deviations from normative lifestyle baselines;
generating, by the one or more processors, utilizing the trained machine-learning model, one or more predictions based upon the one or more patterns, wherein the one or more predictions include (i) one or more dynamically inferred user states, (ii) an estimated progression of behavioral trajectories, or (iii) a probability score for possible interventions; and
selecting, by the one or more processors, utilizing the trained machine-learning model, one or more of the one or more context-aware adaptive responses based upon the one or more predictions.

3. The computer-implemented method of claim 2, wherein the one or more context-aware adaptive responses include (i) an activity suggestion, (ii) one or more personalized health plans, (iii) one or more stress management techniques, (iv) one or more sleep optimization strategies, (v) one or more substance use reduction plans, or (vi) one or more preventive healthcare screenings.

4. The computer-implemented method of claim 1, wherein monitoring the one or more activities performed by the at least one user, further comprises:
receiving, by the one or more processors, one or more real-time data streams from the one or more sensors; and
analyzing, by the one or more processors, the one or more real-time data streams for determining a level of compliance corresponding to the one or more context-aware adaptive responses using a rule-based analysis, wherein the rule-based analysis includes one or more predefined thresholds for the one or more activities to assess the level of compliance.

5. The computer-implemented method of claim 1, wherein updating the avatar based upon the monitored one or more user activities, further comprises:
adjusting, by the one or more processors, an appearance and one or more characteristics of the avatar based upon a condition improvement or a condition deterioration corresponding to the at least one user, wherein the appearance and the one or more characteristics reflect a success or a failure in completing the one or more context-aware adaptive responses.

6. The computer-implemented method of claim 5, further comprising:
modifying, by the one or more processors, one or more facial expressions, a posture, or a clothing item of the avatar to visually indicate the success or the failure,
wherein the success is represented by one or more positive traits of the avatar including (i) an upright posture, (ii) one or more happy facial expressions, or (iii) an athletic clothing item in one or more bright colors, or
wherein the failure is represented by one or more negative traits of the avatar including (i) a slouched posture, (ii) one or more sad facial expressions, or (iii) an untidy clothing item in one or more dark colors.

7. The computer-implemented method of claim 1, wherein the data associated with the at least one user includes (i) behavioral data, (ii) physiological data, (iii) contextual data, (iv) historical data, or (v) health data.

8. The computer-implemented method of claim 1, wherein the one or more sensors include (i) a motion sensor for detecting user movement and physical activity, (ii) a heart rate sensor for detecting pulse and heart rate variability, (iii) a sleep monitoring sensor for determining one or more sleep patterns and a duration, (iv) a temperature sensor for detecting one or more changes in body temperature, (v) a microphone for detecting one or more sounds associated with user behavior, (vi) a geolocation sensor for determining a spatial movement pattern and a location context, or (vii) a pressure sensor embedded in one or more smart clothing items for monitoring a posture and a respiration.

9. The computer-implemented method of claim 1, wherein transforming the received data into the structured format includes (i) normalizing one or more measurement units across multiple data sources to a common scale, (ii) aligning time-series data for consistency across one or more sampling intervals, or (iii) mapping unstructured data into a predefined data schema.

10. The computer-implemented method of claim 1, further comprising:
generating, by the one or more processors, one or more notifications in the user interface, wherein the one or more notifications include (i) one or more rewards, (ii) one or more premium discounts, (iii) one or more achievement-based badges, (iv) one or more goal-setting challenges, or (v) enhancing the avatar to reflect user progress.

11. A computer system for processing data to generate one or more context-aware adaptive responses comprising:
one or more processors of a computing system in communication with one or more data sources; and
at least one non-transitory computer readable medium storing instructions which, when executed by the one or more processors, cause the one or more processors to perform operations comprising:
receiving data associated with at least one user from one or more sensors;
processing the received data to transform the received data into a structured format;
inputting the processed data into a trained machine-learning model for generating the one or more context-aware adaptive responses for the at least one user;
generating a display of the one or more context-aware adaptive responses and a corresponding avatar;
outputting the display and the avatar to a user interface of at least one device associated with the at least one user;
in response to the outputting, monitoring one or more activities performed by the at least one user based upon the display of the one or more context-aware adaptive responses; and
updating the avatar based upon the monitored one or more user activities.

12. The system of claim 11, wherein the trained machine-learning model generating the one or more context-aware adaptive responses, further comprises:
analyzing, utilizing the trained machine-learning model, the processed data to extract one or more statistical features, one or more temporal features, or one or more contextual features;
identifying, utilizing the trained machine-learning model, one or more patterns corresponding to the extracted one or more features, wherein the one or more patterns indicate (i) one or more behavioral profiles, (ii) one or more temporal health state transitions, or (iii) one or more deviations from normative lifestyle baselines;
generating, utilizing the trained machine-learning model, one or more predictions based upon the one or more patterns, wherein the one or more predictions include (i) one or more dynamically inferred user states, (ii) an estimated progression of behavioral trajectories, or (iii) a probability score for possible interventions; and
selecting, utilizing the trained machine-learning model, one or more of the one or more context-aware adaptive responses based upon the one or more predictions.

13. The system of claim 12, wherein the one or more context-aware adaptive responses include (i) an activity suggestion, (ii) one or more personalized health plans, (iii) one or more stress management techniques, (iv) one or more sleep optimization strategies, (v) one or more substance use reduction plans, or (vi) one or more preventive healthcare screenings.

14. The system of claim 11, wherein monitoring the one or more activities performed by the at least one user, further comprises:
receiving one or more real-time data streams from the one or more sensors; and
analyzing the one or more real-time data streams for determining a level of compliance corresponding to the one or more context-aware adaptive responses using a rule-based analysis, wherein the rule-based analysis includes one or more predefined thresholds for the one or more activities to assess the level of compliance.

15. The system of claim 11, wherein updating the avatar based upon the monitored one or more user activities, further comprises:
adjusting an appearance and one or more characteristics of the avatar based upon a condition improvement or a condition deterioration corresponding to the at least one user, wherein the appearance and the one or more characteristics reflect a success or a failure in completing the one or more context-aware adaptive responses.

16. The system of claim 15, further comprising:
modifying one or more facial expressions, a posture, or a clothing item of the avatar to visually indicate the success or the failure,
wherein the success is represented by one or more positive traits of the avatar including (i) an upright posture, (ii) one or more happy facial expressions, or (iii) an athletic clothing item in one or more bright colors, or
wherein the failure is represented by one or more negative traits of the avatar including (i) a slouched posture, (ii) one or more sad facial expressions, or (iii) an untidy clothing item in one or more dark colors.

17. The system of claim 11, wherein the data associated with the at least one user includes (i) behavioral data, (ii) physiological data, (iii) contextual data, (iv) historical data, or (v) health data.

18. A non-transitory computer readable medium for processing data to generate one or more context-aware adaptive responses, the non-transitory computer readable medium storing instructions which, when executed by one or more processors of a computing system in communication with one or more data sources, cause the one or more processors to perform operations comprising:
receiving data associated with at least one user from one or more sensors;
processing the received data to transform the received data into a structured format;
inputting the processed data into a trained machine-learning model for generating the one or more context-aware adaptive responses for the at least one user;
generating a display of the one or more context-aware adaptive responses and a corresponding avatar;
outputting the display and the avatar to a user interface of at least one device associated with the at least one user;
in response to the outputting, monitoring one or more activities performed by the at least one user based upon the display of the one or more context-aware adaptive responses; and
updating the avatar based upon the monitored one or more user activities.

19. The non-transitory computer readable medium of claim 18, wherein the trained machine-learning model generating the one or more context-aware adaptive responses, further comprises:
analyzing utilizing the trained machine-learning model, the processed data to extract one or more statistical features, one or more temporal features, or one or more contextual features;
identifying utilizing the trained machine-learning model, one or more patterns corresponding to the extracted one or more features, wherein the one or more patterns indicate (i) one or more behavioral profiles, (ii) one or more temporal health state transitions, or (iii) one or more deviations from normative lifestyle baselines;
generating utilizing the trained machine-learning model, one or more predictions based upon the one or more patterns, wherein the one or more predictions include (i) one or more dynamically inferred user states, (ii) an estimated progression of behavioral trajectories, or (iii) a probability score for possible interventions; and
selecting utilizing the trained machine-learning model, one or more of the one or more context-aware adaptive responses based upon the one or more predictions.

20. The non-transitory computer readable medium of claim 18, wherein monitoring the one or more activities performed by the at least one user, further comprises:
receiving one or more real-time data streams from the one or more sensors; and
analyzing the one or more real-time data streams for determining a level of compliance corresponding to the one or more context-aware adaptive responses using a rule-based analysis, wherein the rule-based analysis includes one or more predefined thresholds for the one or more activities to assess the level of compliance.
